(19) United States
(12) Patent Application Publication
(10) Pub. No.: US 2024/0407916 A1
(43) Pub. Date: Dec. 12, 2024

(54) PROSTHETIC HEART VALVE FRAMES WITH MARKERS FOR ALIGNMENT

(71) Applicant: MEDTRONIC, INC., Minneapolis, MN (US)

(72) Inventors: Justin PETERSON, Santa Rosa, CA (US); Shahnaz JAVANI, Santa Rosa, CA (US)

(21) Appl. No.: 18/688,785

(22) PCT Filed: Aug. 31, 2022
(86) PCT No.: PCT/IB2022/058185

§ 371 Date: Mar. 3, 2024

Related U.S. Application Data
(60) Provisional application No. 63/240,644, filed on Sep. 3, 2021.

(51) Int. Cl. A61F 2/24; A61M 25/01

(52) U.S. Cl. CPC A61F 2/2418; A61F 2/2427; A61F 2250/0098; A61M 2025/0166

(57) ABSTRACT

A transcatheter valve prosthesis includes a stent having a radially compressed configuration for delivery within a vasculature and an expanded configuration for deployment within a native heart valve. The stent includes an inflow portion, an outflow portion, and at least one commissure post extending between the inflow portion and the outflow portion. Inflow and/or outflow markers are positioned on the stent to enable longitudinal and rotational orientation of the stent during the installation at the implant location.

### FIELD

The present applications is generally related to medical devices and, in particular, to transcatheter heart valve prostheses.

### BACKGROUND

Implantable medical devices, such as stents, scaffolds, and other cardiac intervention devices are utilized to repair or replace problem native biological systems. For example, heart valve replacement in patients with severe valve disease is a common surgical procedure. The replacement can conventionally be performed by open heart surgery, in which the heart is usually arrested and the patient is placed on a heart bypass machine. In recent years, heart valve prostheses have been developed which are implanted using minimally invasive procedures such as transapical or percutaneous approaches. These procedures involve compressing the heart valve prosthesis radially to reduce its diameter, advancing the heart valve prosthesis via a delivery device, such as a catheter, to the correct anatomical position in the heart. Once properly positioned, the heart valve prosthesis is deployed by radial expansion within the native valve annulus.

While these procedures are substantially less invasive than open heart surgery, the lack of line-of-sight visualization of the heart valve prosthesis and the native heart valve presents challenges, because the physician cannot see the orientation of the heart valve prosthesis during the implantation procedure. Correct positioning of the heart valve prosthesis is achieved using radiographic imaging, which yields a two-dimensional image of the viewed area. The physician must interpret the image correctly in order to properly place the prosthetic heart valve in the desired position. Failure to properly position the heart valve prosthesis sometimes leads to migration of the prosthetic heart valve, improper functioning, and or undesired blocking of other native structures, such as, but not limited to, coronary arteries. Proper placement of the heart valve prosthesis using radiographic imaging is thus critical to the success of the implantation.

### SUMMARY

The techniques of this disclosure generally relate to a transcatheter heart valve prosthesis to be implanted at a native heart valve. The transcatheter heart valve prosthesis includes markers to enable proper axial and rotational orientation of the heart valve prosthesis at the native heart valve location.

In a first aspect of the present disclosure, a transcatheter heart valve prosthesis includes a stent having a radially compressed configuration for delivery within a vasculature and an expanded configuration for deployment within a native heart valve. The stent includes an inflow portion comprising a plurality of rows of angled struts, an outflow portion including at least one row of angled struts, and three commissure posts extending between the inflow portion and the outflow portion. The transcatheter heart valve prosthesis further includes three inflow markers positioned within the inflow portion of the stent, wherein each of the three inflow markers is axially aligned with one of the three commissure posts. The inflow markers are configured to be visible relative to the stent in one or more images captured during installation at the native heart valve.

In a second aspect of the disclosure, in the transcatheter heart valve prosthesis of the first aspect, the inflow markers are located at a junction between a first row and second of the plurality of rows of angled struts of the inflow portion.

In a third aspect of the disclosure, in the transcatheter heart valve prosthesis of the first aspect or the second aspect, each of the three inflow markers is spaced a first distance from an inflow end of the stent, the first distance being such that the three inflow markers are configured to be disposed at the annulus of the native heart valve.

In a fourth aspect of the disclosure, in the transcatheter heart valve prosthesis of any one of the first aspect through the third aspect, the stent further includes at least three axial struts extending between the inflow portion and the outflow portion, wherein each one of the three axial struts is located circumferentially between a corresponding pair of the commissure posts such that the commissure posts and the axial struts alternate around a circumference of the stent.

In a fifth aspect of the disclosure, the transcatheter heart valve prosthesis of the fourth aspect further includes a first outflow marker disposed on a first commissure post of the three commissure posts, and a second outflow marker disposed on a first axial strut of the three axial struts, wherein the first axial strut is disposed between the first commissure post and a second commissure post of the three commissure posts adjacent to the first commissure post.

In a sixth aspect of the disclosure, in the transcatheter heart valve prosthesis of the fifth aspect, the second outflow marker is disposed counter-clockwise from the first outflow marker when viewed from outside of the stent.

In a seventh aspect of the disclosure, in the transcatheter heart valve prosthesis of the fifth aspect or the sixth aspect, the second outflow marker is axially offset from the first outflow marker.

In an eighth aspect of the disclosure, in the transcatheter heart valve prosthesis of the seventh aspect, the second outflow marker is disposed upstream of the first outflow marker.

In a ninth aspect of the disclosure, in the transcatheter heart valve prosthesis of the seventh aspect, the second outflow marker is disposed downstream of the first outflow marker.

In a tenth aspect of the disclosure, in the transcatheter heart valve prosthesis of any one of the fifth aspect through the ninth aspect, the second outflow marker is circumferentially offset from the first outflow marker by approximately 60 degrees.

In an eleventh aspect of the disclosure, in the transcatheter heart valve prosthesis of any one of the first aspect through the tenth aspect, the stent further includes at least nine axial struts extending between the inflow portion and the outflow portion, wherein three axial struts are located circumferentially between a corresponding pair of the commissure posts such that there are three axial struts between each pair of commissure posts around a circumference of the stent.

In a twelfth aspect of the disclosure, the transcatheter heart valve prosthesis of the eleventh aspect further includes a first outflow marker disposed on a first commissure post of the three commissure posts and a second outflow marker disposed on a first axial strut of the at least nine axial struts, wherein the first axial strut is disposed between the first commissure post and a second commissure post of the three commissure posts adjacent to the first commissure post.

In a thirteenth aspect of the disclosure, in the transcatheter heart valve prosthesis of the twelfth aspect, the second outflow marker is disposed counter-clockwise from the first outflow marker when viewed from outside of the stent.

In a fourteenth aspect of the disclosure, in the transcatheter heart valve prosthesis of the twelfth aspect or the thirteenth aspect, the second outflow marker is axially offset from the first outflow marker.

In a fifteenth aspect of the disclosure, in the transcatheter heart valve prosthesis of any one of the twelfth through fourteenth aspects, the second outflow marker is disposed upstream of the first outflow marker.

In a sixteenth aspect of the disclosure, in the transcatheter heart valve prosthesis of any one of the twelfth through fourteenth aspects, the second outflow marker is disposed downstream of the first outflow marker.

In a seventeenth aspect of the disclosure, in the transcatheter heart valve prosthesis of any one of the twelfth through sixteenth aspects, the second outflow marker is circumferentially offset from the first outflow marker by approximately 60 degrees.

In an eighteenth aspect of the disclosure, a method for rotationally aligning a transcatheter heart valve prosthesis within a native heart valve includes percutaneously delivering the transcatheter heart valve prosthesis to the native heart valve, wherein the transcatheter heart valve prosthesis includes at least one marker substantially aligned with a commissure of the transcatheter heart valve prosthesis. The method further includes receiving an image of the transcatheter heart valve prosthesis within the native heart valve, determining, based on the image and the at least one marker, whether the transcatheter heart valve prosthesis is in a desired rotational orientation, and if the at least one marker in the viewing angle image indicates that the transcatheter heart valve prosthesis is not in the desired rotational orientation, rotating the transcatheter heart valve prosthesis until the transcatheter heart valve prosthesis is in the desired rotational orientation.

In a nineteenth aspect of the disclosure, in the method of the eighteenth aspect, the at least one marker is disposed adjacent an inflow end of the transcatheter heart valve prosthesis.

In a twentieth aspect of the disclosure, in the method of the eighteenth aspect or the nineteenth aspect, the viewing angle is a cusp overlap viewing angle and the image is a cusp overlap viewing angle image.

In a twenty-first aspect of the disclosure, in the method of the twentieth aspect, the at least one marker comprises three inflow markers with each inflow marker substantially aligned with a commissure of a valve structure of the transcatheter valve prosthesis, and determining whether the transcatheter heart valve prosthesis is in the desired rotational orientation comprises determining, based on the cusp overlap viewing angle image and the three inflow markers, whether two of the inflow markers are left of a centerline of the cusp overlap viewing angle image.

In a twenty-second aspect of the disclosure, in the method of the eighteenth aspect or the nineteenth aspect, the view angle is a coronary overlap viewing angle and the image is a coronary overlap viewing angle image.

In a twenty-third aspect of the disclosure, in the method of the twenty-second aspect, the at least one marker comprises three inflow markers with each inflow marker substantially aligned with a commissure of a valve structure of the transcatheter valve prosthesis, and determining whether the transcatheter heart valve prosthesis is in the desired rotational orientation comprises determining, based on the coronary overlap viewing angle image and the three inflow markers, whether two of the inflow markers are left of a centerline of the coronary overlap viewing angle image.

In a twenty-fourth aspect of the disclosure, in the method of the eighteenth aspect or the nineteenth aspect, the viewing angle is a three cusp viewing angle and the image is a three cusp viewing angle image.

In a twenty-fifth aspect of the disclosure, in the method of the twenty-fourth aspect, the at least one marker comprises a first outflow marker substantially aligned with a commissure of a valve structure of the transcatheter valve prosthesis and a second outflow marker circumferentially offset and axially offset from the first outflow marker, and determining whether the transcatheter heart valve prosthesis is in the desired rotational orientation comprises determining, based on the three cusp view angle image, whether the first outflow marker and the second outflow marker are stacked on a right side of the three cusp overlap viewing angle image.

In a twenty-sixth aspect of the disclosure, in the method of the twenty-fifth aspect, the second outflow marker is circumferentially offset from the first outflow marker by approximately 60 degrees.

In a twenty-seventh aspect of the disclosure, in the method of the twenty-fifth aspect or the twenty-sixth aspect, the second outflow marker is upstream of the first outflow marker.

In a twenty-eighth aspect of the disclosure, in the method of the twenty-fifth aspect or the twenty-sixth aspect, the second outflow marker is downstream of the first outflow marker.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIG. 1 illustrates a transcatheter heart valve prosthesis in an expanded configuration in accordance with an embodiment hereof.

FIG. 2 illustrates a partial view of the stent of transcatheter heart valve prosthesis of FIG. 1.

FIG. 3 illustrates a laid open, flat view of the stent of the transcatheter heart valve prosthesis of FIG. 1.

FIGS. 4A and 4B depict illustrations of a native aortic valve as viewed from the aorta.

FIG. 5A depicts an illustration of a fluoroscopic image using a three cusp viewing angle with the transcatheter heart valve prosthesis of FIG. 1 in the native aortic valve.

FIG. 5B depicts illustrations of a fluoroscopic image using a three cusp viewing angle and movement of markers of the transcatheter heart valve prosthesis of FIG. 1 as the prosthesis is rotated.

FIG. 6A depicts an illustration of the native aortic valve as viewed from the aorta and including markers of a transcatheter heart valve prosthesis.

FIG. 6B illustrates a schematic representation of a fluoroscopic image of a native aortic valve using the cusp overlap view and showing markers of a transcatheter heart valve prosthesis disposed therein.

FIG. 7A depicts an illustration of the native aortic valve as viewed from the aorta and including markers of a transcatheter heart valve prosthesis.

FIG. 7B illustrates a schematic representation of a fluoroscopic image of a native aortic valve using the coronary overlap view and showing markers of a transcatheter heart valve prosthesis disposed therein in a radially compressed delivery configuration.

FIG. 7C illustrates a schematic representation of a fluoroscopic image of a native aortic valve using the coronary overlap view with the transcatheter heart valve prosthesis shown in FIG. 7B in a radially expanded, deployed configuration.

FIG. 8 illustrates a laid open, flat view of a stent of a transcatheter heart valve prosthesis including markers in accordance with another embodiment hereof.

FIG. 9A illustrates a transcatheter heart valve prosthesis in an expanded configuration in accordance with another embodiment hereof.

FIG. 9B illustrates a laid open, flat view of the stent of the transcatheter heart valve prosthesis of FIG. 9A.

FIGS. 10A and 10B depict illustrations of a native aortic valve as viewed from the aorta.

FIG. 11 depicts an illustration of a fluoroscopic image using a three cusp viewing angle with the transcatheter heart valve prosthesis of FIG. 1 in the native aortic valve.

FIG. 12A depicts an illustration of the native aortic valve as viewed from the aorta and including markers of a transcatheter heart valve prosthesis.

FIG. 12B illustrates a schematic representation of a fluoroscopic image of a native aortic valve using the cusp overlap view and showing markers of a transcatheter heart valve prosthesis disposed therein.

FIG. 13A depicts an illustration of the native aortic valve as viewed from the aorta and including markers of a transcatheter heart valve prosthesis.

FIG. 13B illustrates a schematic representation of a fluoroscopic image of a native aortic valve using the coronary overlap view and showing markers of a transcatheter heart valve prosthesis disposed therein.

FIG. 14 illustrates a laid open, flat view of a stent of a transcatheter heart valve prosthesis including markers in accordance with another embodiment hereof.

### DETAILED DESCRIPTION

Specific embodiments of the present disclosure are now described with reference to the figures, wherein like reference numbers indicate identical or functionally similar elements.

The following detailed description describes examples of embodiments and is not intended to limit the present technology or the application and uses of the present technology. Although the description of embodiments hereof is in the context of transcatheter heart valve prosthesis, the present technology may also be used in other devices. Furthermore, there is no intention to be bound by any expressed or implied theory presented in the preceding technical field, background, brief summary or the following detailed description.

The terms “distal” and “proximal”, when used in the following description to refer to a delivery system or catheter are with respect to a position or direction relative to the treating clinician. Thus, “distal” and “distally” refer to positions distant from, or in a direction away from the treating clinician, and the terms “proximal” and “proximally” refer to positions near, or in a direction toward the clinician. The terms “distal” and “proximal”, when used in the following description to refer to a device to be implanted into a vessel, such as a heart valve prosthesis, are used with reference to the direction of blood flow. Thus, “distal” and “distally” refer to positions in a downstream direction with respect to the direction of blood flow, and the terms “proximal” and “proximally” refer to positions in an upstream direction with respect to the direction of blood flow.

Embodiments of disclosed herein are directed to a transcatheter heart valve prosthesis with markers for positioning and orienting the transcatheter heart valve prosthesis. The markers are positioned on the transcatheter heart valve prosthesis such that the markers provide a visual indication that the transcatheter heart valve prosthesis is properly oriented. The markers can include radiopaque materials that are visible on radiographic imaging systems.

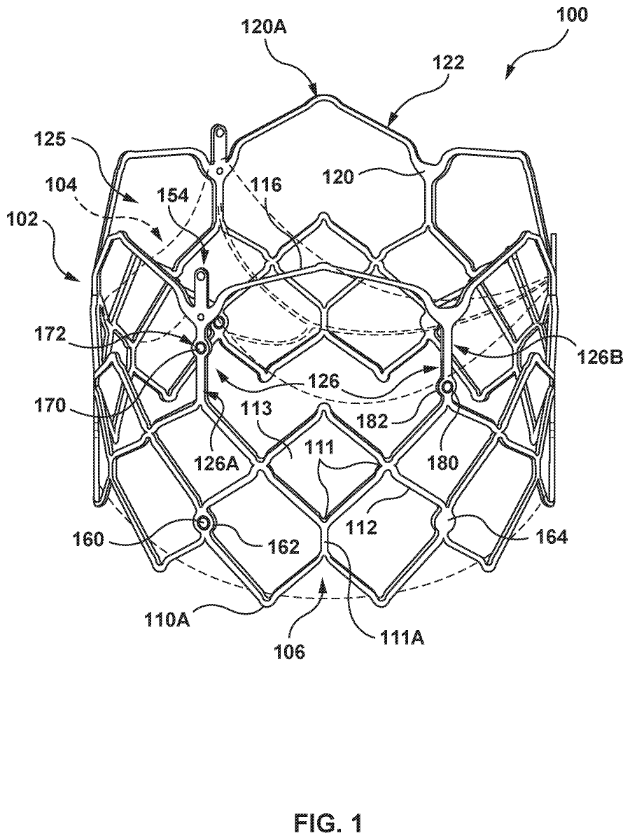

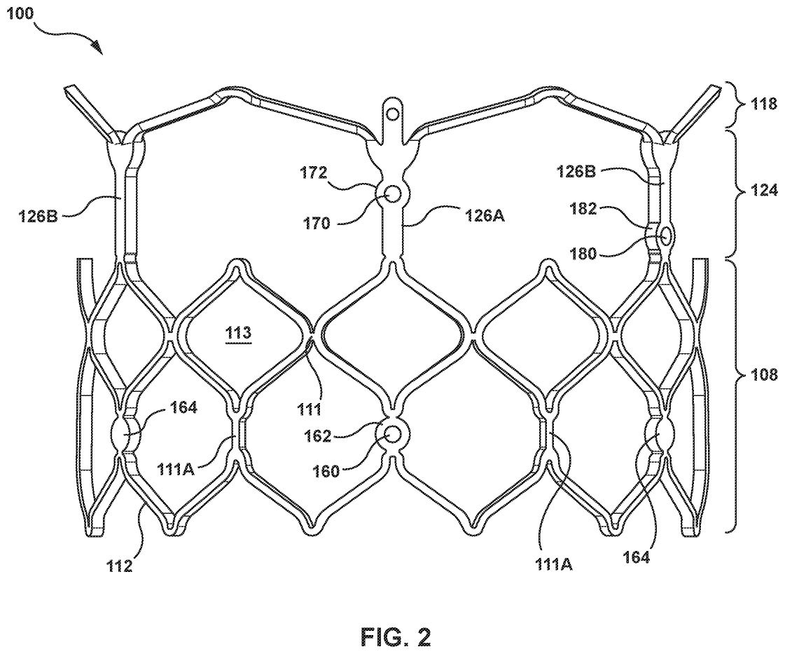

FIGS. 1-3 illustrate a transcatheter heart valve prosthesis 100 including radially-expandable frame or stent 102, a prosthetic valve 104 coupled to the stent 102, three inflow markers 160, and two outflow markers 170, 180, as described in greater detail below. The prosthetic valve 104 is shown in dashed lines for clarity. In embodiments, the inflow markers 160 and the outflow markers 170, 180 can be utilized in orientation (e.g., axial alignment, tilt alignment, circumferential (rotational) alignment, etc.) of the transcatheter heart valve prosthesis 100, in situ, as discussed in detail below.

One skilled in the art will realize that FIGS. 1-3 illustrate one example of an implantable medical device and that existing components illustrated in FIGS. 1-3 may be removed and/or additional components may be added. For example, and not by way of limitation, inner and outer skirts may be included in the transcatheter heart valve prosthesis 100.

The stent 102 has a non-expanded or crimped or radially compressed configuration (not shown), and a radially expanded configuration, which is shown FIG. 1. “Non-expanded” or “crimped” or “radially compressed” configuration as used herein refer to the configuration of the stent 102 after crimping. For example, the stent 102 can be crimped onto a balloon of a balloon catheter for delivery. In some embodiments, the stent 102 can be mechanically or balloon expandable. As such, the stent 102 can be made from a plastically deformable material such that, when expanded by a dilatation balloon, the stent 102 maintains its radially expanded configuration after balloon deflation. The stent 102 can be formed from stainless steel or other suitable metal, such as platinum iridium, cobalt chromium alloys such as MP35N, or various types of polymers or other materials known to those skilled in the art, including said materials coated with various surface deposits to improve clinical functionality. In some embodiments, the stent 102 can be self-expanding.

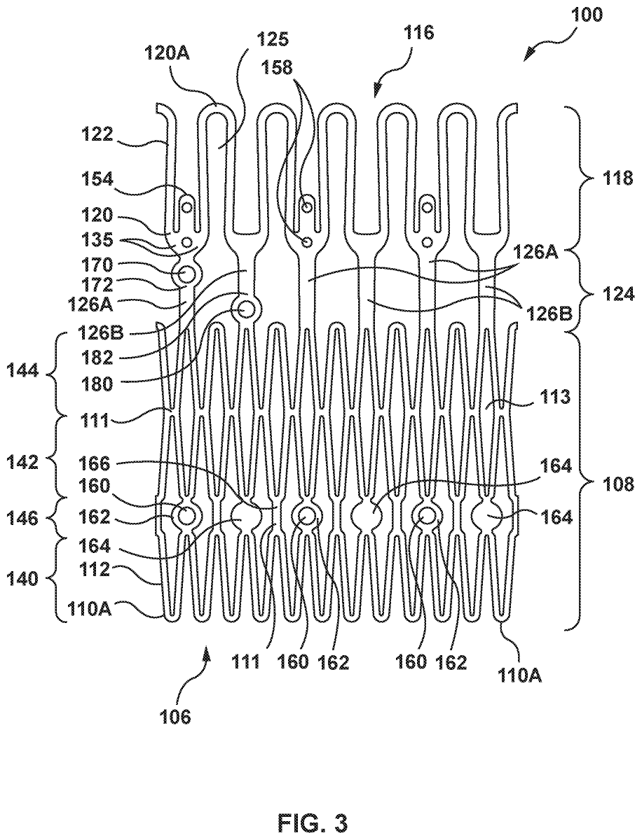

In embodiments, the stent 102 includes an inflow portion 108, an outflow portion 118, and a transition portion 124 bridging, connecting, or otherwise extending between the inflow portion 108 and the outflow portion 118. The stent 102 can be a generally tubular component defining a central lumen or passageway and can have an inflow or proximal end 106 and an outflow or distal end 116. In some embodiments, when expanded, a diameter of the inflow end 106 of the stent 102 can be the same as a diameter of the outflow end 116 of the stent 102. The stent 102 can be formed by a laser-cut manufacturing method and/or another conventional stent forming method as would be understood by one of ordinary skill in the art. The transverse cross-section of the stent 102 can be trapezoidal, circular, ellipsoidal, rectangular, hexagonal, square, or other polygonal shape, although at present it is believed that trapezoidal, circular or ellipsoidal may be preferable when utilized with the replacement of an aortic valve. FIG. 3 shows an open, flat view of an example of the stent 102.

The stent 102 can be configured to be rigid such that it does not deflect or move when subjected to in-vivo forces, or such that deflection or movement is minimized when subjected to in-vivo forces. In an embodiment, the radial stiffness (i.e., a measurement of how much the stent 102 deflects when subjected to in-vivo forces) of the stent 102 can be between 80 N/m and 120 N/m, and the radial stiffness of the stent 102 scaled across the deployed height thereof is approximately 5 N/mm². In an embodiment, the radial stiffness of the stent 102 can be greater than 100 N/m. Further, in an embodiment, the device recoil (i.e., a measurement of how much the stent 102 relaxes after balloon deployment) can below 15% and the approximate recoil after deployment is between 0.5 mm and 2 mm. Further, in an embodiment, the device crush or yield (i.e., the radial force at which the stent 102 yields) can be approximately 200 N. While the above describes examples of radial stiffness for the stent 102, one skilled in the art will realize that the stent 102 may have any radial stiffness as required by a given application and/or governed by the design and construction of the stent 102.

The prosthetic valve 104 is disposed within and secured to at least the transition portion 124 of the stent 102, as illustrated in FIG. 1 In addition, the prosthetic valve 104 can also be disposed within and secured to the inflow portion 108 of the stent 102 and/or the outflow portion 118 of the stent 102. One skilled in the art will realize that the prosthetic valve 104 can be disposed within and secured to one or more of the inflow portion 108, outflow portion 118, or the transition portion 124, for example, depending on the design and construction of the prosthetic valve 104 and/or the design and construction of the stent 102. Further, in some embodiments, the prosthetic valve 104 is secured to a skirt, such as an inner skirt, which is in turn secured to the stent 102, such as at the inflow portion 108, the transition portion 124, or the outflow portion 118.

The inflow portion 108 can be formed proximate to the inflow end 106 of the stent 102. The inflow portion 108 of the stent 102 may be formed with struts 112 and crowns or bends 110 coupling circumferentially adjacent struts to each other. The struts 112 may also be referred to as angled struts in that the angled struts 112 are not parallel to the central longitudinal axis of the stent 102. The struts 112 of the inflow portion 108 may also be referred to as inflow struts. The crowns 110 of the inflow portion 108 may also be referred to as inflow crowns. Nodes 111 are formed at intersections of a crowns 110 of adjacent rows of struts and crowns. In some embodiments, the inflow end 106 of the stent 102 can include a total of twelve endmost inflow crowns 110A. Cells 113 of the inflow portion 108 of the stent 102 are defined by a pair of circumferentially adjacent struts 112 of a row of struts and crowns, a pair of circumferentially adjacent struts 112 of an adjacent row of struts and crowns, and nodes 111 between the adjacent rows of struts and crowns. Cells 113 define open spaces in the inflow portion 108 of the stent 102.

The outflow portion 118 can be formed proximate to the outflow end 116 of the stent 102. The outflow portion 118 can be configured in a shape that forms a central lumen or passageway, for example, a ring. The outflow portion 118 can include a plurality of crowns 120 and a plurality of struts 122 with each crown 120 being formed between a pair of opposing struts 122. Each crown 120 can be a curved segment or bend extending between opposing struts 122. The struts 122 of the outflow portion 118 may also be referred to as angled struts and/or outflow struts. The crowns 120 of the outflow portion 118 may also be referred to as outflow crowns. A series of endmost outflow crowns 120A are formed at the outflow end 116 of the stent 102. In some embodiments, the outflow end 116 of the stent 102 can have a total of six endmost outflow crowns 120A.

The transition portion 124 bridges, connects, or otherwise extends between the inflow portion 108 and the outflow portion 118. In some embodiments, the transition portion 124 can include a minimum of three axial frame members 126, each axial frame member 126 extending between a crown 120 of the outflow portion 118 and a crown 110 of the inflow portion 108. Each axial frame member 126 can be connected to a crown 120 of the outflow portion 118 and connected to a crown 110 of the inflow portion 108. The axial frame members 126 can be substantially parallel to the central longitudinal axis of the stent 102. “Substantially parallel” as used herein when referring to the axial frame members 126 means that the axial frame members 126 are within 10 degrees of parallel to the central longitudinal axis of the stent. Each axial frame member 126 can be disposed circumferentially approximately halfway between a pair of adjacent endmost outflow crowns 120A. While the stent 124 has been described as including a transition portion 124, one skilled in the art will realize that the transition portion 124 may form a portion of the inflow portion 108 and/or the outflow portion 118.

In an embodiment, the transition portion 124 can include up to six axial frame members 126, with three of the axial frame members 126 being commissure posts 126A and three of the axial frame members 126 being axial struts 126B. The commissure posts 126A and the axial struts 126B can be being alternatingly positioned, as illustrated, for example, in FIG. 3. The commissure posts 126A can be circumferentially spaced apart and aligned with and attached to a respective commissure of three leaflets of the prosthetic valve 104, and the axial struts 126B can be disposed between adjacent commissure posts 126A. The axial frame members 126 may aid in valve alignment and coaptation. More particularly, the axial frame members 126 reinforce or strengthen the commissure region of the prosthetic valve by shaping the leaflets and supporting the leaflets during opening and closing thereof, and thus provide more reliable leaflet coaptation. In addition, the axial frame members 126 maximize symmetrical cell expansion. Other embodiments may include more than six axial frame members 126. For example, and not by way of limitation, in embodiments with twelve endmost outflow crowns 120A, there may be twelve axial frame members 126, with three being commissure posts 126A and nine being axial struts 126B.

In an embodiment, the endmost outflow crowns 120A are not connected to the axial frame members 126 but rather may be considered to be free or unattached while the remaining outflow crowns 120 of the outflow portion 118 adjacent the transition portion 124 are connected to the axial frame members 126 and disposed closer to the inflow end 106 than the endmost outflow crowns 120A. In the embodiment shown, the stent 102 includes a single row of struts 122 and crowns 120 of the outflow portion coupled to the axial frame members 126 and defining the outflow end 116 of the stent 102. Further, in the embodiment shown, exactly two struts 122 and a single crown 120 of the outflow portion 118 are disposed between adjacent axial frame members 126. Such an arrangement can provide a series of six endmost cells 125 formed at the outflow portion 118 of the stent 102.

In embodiments, each endmost cell 125 can define an open space in the stent 102, which is formed in any type of shape, in the radially expanded configuration, for example, as shown in FIG. 1. Each endmost cell 125 can be defined by two circumferentially adjacent struts 122 of the outflow portion 118, the endmost crown 120A connecting the struts 122, four circumferentially adjacent struts 112 of the inflow portion 108, the crowns 110 connecting the struts 112, and two circumferentially adjacent axial frame members 126 of the transition portion 124. The endmost cells 125 of the outflow portion 118 can be relatively larger than the cells 113 of the inflow portion 108 to improve access to the coronary arteries when used for aortic valve replacement. More particularly, the endmost cells 125 of the outflow portion 118 can be configured to be of sufficient size to be easily crossed with a coronary guide catheter into either the right coronary artery or the left main coronary artery once the transcatheter valve prosthesis 100 is deployed, in situ.

In an embodiment, the inflow portion 108 can include exactly three rows of struts 112 and crowns 110 between the inflow end of the axial frame members 126 and the inflow end 106 of the stent 102. The rows may also be referred to as rows of angled struts. In particular, as illustrated in FIG. 3, the stent 102 can include a first row 140 of the struts 112 and crowns 110 formed proximate to the inflow end 106, a second row 142 of the struts 112 and crowns 110 formed longitudinally adjacent the first row 140 and towards the outflow end 116 of the stent 102 relative to the first row 140, and a third row 144 of struts 112 and crowns formed longitudinally adjacent the second row 142 and towards the outflow end 116 of the stent 102 relative to the second row 142. The inflow portion 108 further includes an elongated junction 146 between the first row 140 and the second row 142. As can be seen in FIG. 3, the junction 146 is longitudinally elongated relative the nodes 111 formed between the second row 142 and the third row 144.

Further, in this embodiment, four struts 112 and three crowns 110 can be disposed circumferentially between adjacent axial frame members 126. One skilled in the art will realize that the above configuration of the inflow portion 108 is one example of a configuration of the inflow portion 108 and that the inflow portion 108 can include fewer or additional rows of struts 112 and crowns 110. Likewise, one skilled in the art will realize that each row can include fewer or additional numbers of struts 112 and crowns 110.

In an embodiment, a height or length of the stent 102 in the expanded configuration can be between 14 and 23 mm, the height being measured from the most proximal part thereof to the most distal part thereof, and a diameter of the stent 102 in the expanded configuration can be between 18 and 31 mm. For example, an expanded 21 mm diameter device may be 15 mm in height. An expanded 30 mm diameter device may have a 21 mm height. Additionally, one skilled in the art will realize that the ranges of the height and diameter of the stent 102 are examples and that the height and diameter of the stent 102 may vary based on an amount of expansion of the stent 102, for example, as required by a given application and/or governed by the design and construction of the stent 102.

In an embodiment, the axial frame members 126 can include commissure posts 126A that are formed to have an axial length greater than the axial struts 126B. In this embodiment, a first or inflow end of each of the axial struts 126B, which is closer to the inflow end 106, can be coupled to a pair of struts 112 of the inflow portion 108 or a crown 110 of the inflow portion 108. A second or outflow end of each of the axial struts 126B, which is closer to the outflow end 116, can be coupled to a pair of the struts 122 of the outflow portion 118. A first or inflow end of each of the commissure posts 126A, which is closer to the inflow end 106, can be coupled to a pair of the struts 112 of the inflow portion or a crown 110 of the inflow portion. Because the commissure posts 126A are longer than the axial struts 126B, pairs of struts 122 of the outflow portion 118 are coupled to the commissure posts 126A at side portions 155 of the commissure posts 126A (e.g., an intersection of the commissure posts 126A and the struts 122). The location of the connection to the side portions 155 is spaced a distance, in the direction of the inflow end 106, from the second or outflow end of the commissure posts 126A, which is closer to the outflow end 116. In other words, each commissure posts 126A can be a relatively stiff, axial segment or planar bar having the inflow end connected to a pair of struts 112 at a crown 110 of the inflow portion 108 and having an unattached or free outflow end distal of the side portions 155. As such, the connection of the struts 122 to the side portions 155 defines an outflow portion 154 of each commissure post 126A, which extends from the side portions 154 to the outflow end of the commissure post 126A, and is positioned in the outflow portion 118.

The outflow portions 154 can be configured as support features that allow for lengthened commissure posts 126A to further reinforce or strengthen the commissure region of the transcatheter valve prosthesis 100. Each of the outflow portions 154 can extend into the outflow portion 118 of the stent 102 to allow for lengthened commissure posts 126A without increasing the overall height of the transcatheter valve prosthesis 100. In an embodiment, the stent 102 can include a total of three commissure posts 126A, which include three outflow portions 154. The commissure posts 126A, which include the outflow portions 154, one for each commissure post 126A, can extend substantially parallel to the central longitudinal axis of the stent 102 and are circumferentially spaced apart from each other. The commissure posts 126A, which include the outflow portions 154, can include holes or openings 158 formed therein configured to attach a respective commissure of the three leaflets of the prosthetic valve to the stent 102. Additionally, in some embodiments, the commissure posts 126A, which include the outflow portions 154, can include one or more holes or openings to support alignment markers, as described further below. One skilled in the art will realize that the above configuration of the outflow portions 154 is one example of a configuration and that the stent 102 can include additional outflow portions 154 based on the design or configuration of the stent 102.

As discussed above, the commissure posts 126A can be formed to be lengthened relative to the axial struts 126B. The commissure posts 126A can reduce stresses observed at the commissure region during valve loading by spreading out such stresses across a larger area. More particularly, as compared to self-expanding valve stents, balloon expandable valve stents are stiffer and stronger but therefore may place more stress on the valve leaflets attached to the stent 102. The valve leaflets, which are often formed from tissue, are more durable when the portion of the stent to which they are attached is more flexible, but such stent flexibility may be detrimental to stent fatigue. As such, the commissure posts 126A achieve a balance between stent durability and tissue durability because the stent 102 maintains its strength and durability while the lengthened commissure posts improve or increase tissue durability of the valve leaflets by stress relief due to the lengthened commissure posts.

Further, the performance of the transcatheter valve prosthesis 100 may be enhanced by the lengthened commissure posts 126A without increasing the overall height of the transcatheter valve prosthesis 100. For example, in the unexpanded or compressed configuration, as illustrated in FIG. 1A, the outflow portions 154 of the commissure posts 126A extend into the outflow portion 118, but do not extend beyond the endmost outflow crowns 120A. In the expanded or uncompressed configuration, as illustrated in FIG. 1C, the outflow portions 154 of the commissure posts 126A extend into the outflow portion 118, but do not extend beyond the endmost outflow crowns 120A. In other words, the length of the commissure post 126A is increased without increasing the length of the transition portion 124 and the overall height of the transcatheter valve prosthesis 100. A relatively short or minimized overall height can be desirable to increase coronary access and improve system deliverability. In another embodiment hereof (not shown), the stent 100 can include additional commissure posts 126A, which include the outflow portions 154. Inclusion of additional commissure posts 126A, which include the outflow portions 154, may aid in valve alignment and coaptation. In other embodiments, the outflow portions 154 are not included.

In embodiments, the transcatheter valve prosthesis 100 can be configured to allow the inflow cells 113 to expand symmetrically. Symmetrical cell expansion may ensure that the stent 102 crimps well onto a balloon of a balloon catheter for delivery. This can address poor crimp quality that may lead to portions of the stent 102 overlapping when crimped, which in turn may cause tissue damage to the valve leaflets of the prosthetic valve during the crimping process.

In embodiments, to ensure the proper placement in the native anatomy of a subject, the transcatheter valve prosthesis 100 can include the inflow markers 160 and outflow markers 170, 180, as described in more detail below. The inflow markers 160 can be used for depth (longitudinal) alignment and rotational alignment of the stent 102, as described in more detail below. The outflow markers 170, 180 may be used for rotational alignment and can operate as a guide for determining a front or rear location the outflow markers 170, 180 in 2D images during implantation, as described below. The outflow markers 170, 180 may also be referred to as a first outflow marker 170 and a second outflow marker 180.

In an embodiment, as shown in FIGS. 1-3, the first outflow marker 170 can be positioned on a commissure post 126A and the second outflow marker 180 can be positioned on an axial strut 126B that is circumferentially adjacent to the commissure post 126A on which the outflow marker 180 is positioned. Further, as shown in FIGS. 1-3, the second outflow marker 180 can be positioned closer to the inflow end 106 of the stent 102 than the first outflow marker 170 is to the inflow end 106 of the stent 102. In other words, the second outflow marker 180 is positional proximally or upstream relative to the first outflow marker 170. Stated another way, the first outflow marker 170 is positioned adjacent the outflow end of a commissure post 126A and the second outflow marker 180 is positioned adjacent the inflow end of an axial strut 126B. In other embodiments, the longitudinal orientation of the first and second outflow markers 170, 180 may be reversed such that the first outflow marker 170 is proximal to or upstream of the second outflow marker 180.

In embodiments, the outflow markers 170, 180 can be attached to the stent 102 within containment members 172, 182 formed in a commissure post 126A and an axial strut 126B, respectively. The containment members 172, 182 can be positioned at locations along the commissure post 126A and the axial strut 126 as described above for locating the outflow markers 170, 180. Details of the containment members may be found in U.S. patent application Ser. No. 17/187,261, filed Feb. 26, 2021, which is incorporated by reference herein in its entirety. In other embodiments, the outflow markers 170, 180 need not be located within containment members 172, 182. For example, and not by way of limitation, the outflow markers 170, 180 may comprise radiopaque bands that are attached to the commissure post 126A and the axial strut 126B, respectively. In another example, and not by way of limitation, the outflow markers 170, 180 may be formed by applying radiopaque materials to the commissure post 126A and the axial strut 126B, respectively, in any shape. One skilled in the art will realize that the outflow markers 170, 180 may be attached to or formed on the stent 102 utilizing any processes as required by the design of the stent 102 and/or application of the transcatheter valve prosthesis 100.

In any embodiment, the first and second outflow markers 170, 180 can be formed to dimensions such that the first and second outflow markers 170, 180 do not adversely affect the operation of the transcatheter valve prosthesis 100. For example, the first and second outflow markers 170 can be formed to not extend beyond the exterior diameter of the stent 102 or extend into the central lumen of the stent 102. In other words, it is preferable for the first outflow marker 170 to have a radial depth that is equal to or less than the radial depth of the commissure post 126A and for the second outflow marker 180 to have a radial depth that is equal to or less than the radial depth of the axial strut 126B. In an embodiment, the first and second outflow markers 170, 180 may each have a circular cross-sectional shape with a diameter ranging between approximately 0.5 mm and 1.0 mm, for example, approximately 0.8 mm. In such an embodiment, the containment members 172, 182 can each have a circular cross-sectional shape with a diameter ranging between approximately 0.5 mm and 1.0 mm, for example, approximately 0.8 mm. In another embodiment, the first and second outflow markers 170, 180 can have an elliptical cross-sectional shape with an axial diameter ranging between approximately 0.5 mm and 1.0 mm, for example, approximately 0.8 mm, and a circumferential diameter ranging between approximately 0.5 mm and 1.0 mm, for example, approximately 0.9 mm. In this embodiment, the containment members 172, 182 can each have an elliptical cross-sectional shape with an axial diameter ranging between approximately 0.5 mm and 1.0 mm, for example, approximately 0.8 mm, and a circumferential diameter ranging between approximately 0.5 mm and 1.0 mm, for example, approximately 0.9 mm.

In the embodiment of FIGS. 1-3, the stent 102 includes three inflow markers 160. The inflow markers 160 are each positioned towards the inflow end 106 of the stent 102 in the inflow portion 108. In an embodiment, as illustrated in FIGS. 1-3, the inflow markers 160 are positioned between the first row 140 of angled struts and the second row 142 of angled struts in the inflow portion, in the elongated junction 146 of the inflow portion 108. As also shown in FIGS. 1-3, the inflow markers 160 are axially aligned with the commissure posts 126A. In other words, each inflow marker 160 is located along a common longitudinal axis with one of the commissure posts 126A. Stated another way, each inflow marker 160 is located “below” one of the commissure posts 126A.

The elongated junction 146 of the inflow portions 108 also includes axial struts 166 at some nodes 111 and disks 164 at other nodes 111. Thus, as best shown in FIG. 3, the elongated junction 146 includes: three inflow markers 160, with each one axially aligned with a respective commissure post 126A; three disks 164, with each one axially aligned with a respective one of the axial struts 126B; and three struts 166, with each one positioned circumferentially between one of the inflow markers 160 and one of the disks 164. The axial struts 166 are utilized to maintain spacing between the first row 140 of struts and the second row 142 of struts created by the markers 160 in containment members 162 and the disks 164. Those skilled in the art would recognize that if different inflow markers are used, the disks 164 and axial struts 166 may be modified or eliminated depending on the type, size, and/or location of the inflow markers. Further, those skilled in the art would recognize that the disks 164 may be replaced with axial struts 166, or vice versa. The inflow markers 160 may be positioned longitudinally at a location on the stent 102 that is desired to be aligned longitudinally with the aortic annulus or the basal plane, as explained in more detail below.

In embodiments, each inflow marker 160 can be attached to the stent 102 within a respective containment member 162. Details of the containment members 162 may be found in U.S. patent application Ser. No. 17/187,261, filed Feb. 26, 2021, which is incorporated by reference herein in its entirety. In other embodiments, the inflow marker 160 need not be located within containment members 162. For example, and not by way of limitation, the inflow markers 160 may comprise radiopaque bands that are attached to the inflow portion 118. In another example, and not by way of limitation, the inflow markers 160 may be formed by applying radiopaque materials to the inflow portion 118 in any shape. One skilled in the art will realize that the inflow markers 160 may be attached to or formed on the stent 102 utilizing any processes as required by the design of the stent 102 and/or application of the transcatheter valve prosthesis 100.

In embodiments, the inflow marker 160 can be formed to dimensions such that the inflow markers 160 do not adversely affect the operation of the transcatheter valve prosthesis 100. For example, the inflow marker 160 can be formed to not extend beyond the exterior diameter of the stent 102 or extend into the central lumen of the stent 102, e.g., having a radial depth that is equal to or less than the radial depth of the struts 112 of the inflow portion. In an embodiment, the inflow markers 160 can have a circular cross-sectional shape with a diameter ranging between approximately 0.5 mm and 1.0 mm, for example, approximately 0.8 mm. In this embodiment, the containment members 162 can have a circular cross-sectional shape with a diameter ranging between approximately 0.5 mm and 1.0 mm, for example, approximately 0.8 mm. In another embodiment, the inflow markers 160 can have an elliptical cross-sectional shape with an axial diameter ranging between approximately 0.5 mm and 1.0 mm, for example, approximately 0.8 mm, and a circumferential diameter ranging between approximately 0.5 mm and 1.0 mm, for example, approximately 0.9 mm. In this embodiment, the containment members 162 can have an elliptical cross-sectional shape with an axial diameter ranging between approximately 0.5 mm and 1.0 mm, for example, approximately 0.8 mm, and a circumferential diameter ranging between approximately 0.5 mm and 1.0 mm, for example, approximately 0.9 mm.

In any embodiment, the inflow markers 160 and/or the outflow markers 170, 180 can be formed in any shape to assist in the alignment of the transcatheter valve prosthesis 100. In embodiments, as illustrated in FIGS. 1-3, the inflow markers 160 and/or the outflow markers 170, 180 can be formed having a circular cross-sectional shape. In other embodiments, the inflow markers 160 and/or the outflow markers 170, 180 can be formed in any other 2D or 3D shape, which has any type of 2D or 3D cross-sectional shape, such as pins, dots, ovals, spheres, triangles, cones, squares, cubes, bars, crosses, bands, rings, letters, and combination thereof. One skilled in the art will realize that other configurations and shapes of the inflow markers 160 and/or the outflow markers 170, 180 may be provided to provide a benefit for a given application.

In any embodiment, the inflow markers 160 and/or the outflow markers 170, 180 include radiopaque or other material that allow the inflow markers 160 and the outflow markers 170, 180 to be detected and/or viewed via fluoroscopy during the installation of the transcatheter valve prosthesis 100. Examples of radiopaque materials include metals, e.g., stainless steel, titanium, tungsten, tantalum, gold, platinum, platinum-iridium, and/or other polymeric materials, e.g., nylon, polyurethane, silicone, PEBAX, PET, polyethylene, that have been mixed or compounded with compounds of barium, bismuth and/or zirconium, e.g., barium sulfate, zirconium oxide, bismuth sub-carbonate, etc.

In embodiments, the inflow markers 160 and the outflow markers 170, 180 can be utilized to orient (e.g., axial alignment, tilt alignment, circumferential (rotational) alignment, etc.) the transcatheter valve prosthesis 100, in situ, during installation as described below. In particular, as noted briefly above, when installing the transcatheter heart valve prosthesis 100, it is desirable to properly align the stent 102 within the target site. For example, the transcatheter heart valve prosthesis 100 needs to be properly aligned axially, so that the transcatheter valve prosthesis 100 properly engages the native leaflets/tissue of the target site, e.g., the aortic annulus, without causing conduction blockages by implanting too deep or causing an embolization of the transcatheter heart valve prosthesis 100 by implanting too high. Likewise, the transcatheter valve prosthesis 100 needs to be aligned circumferentially or rotationally. When being positioned, in situ, it is very important to avoid blocking the right coronary artery ostium RCO and/or the left main coronary artery ostium LCO. Proper circumferential or rotational orientation within the target site reduces the risk of blocking coronary access. Further, proper rotational alignment provides alignment or near alignment of the prosthetic valve commissures with the native valve commissures. Commissural alignment may improve, for example, coronary flow and leaflet washout.

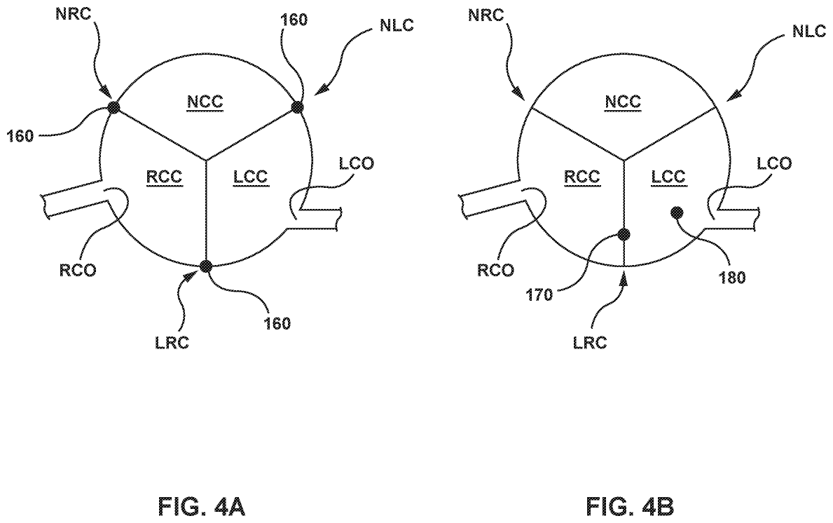

FIGS. 4A-4B show an example of an idealized native aortic valve (e.g. the native commissures are 120 degrees apart) as would be viewed from the aorta, also referred to as the normal or perpendicular view. As shown in FIGS. 4A-4B, the native aortic valve includes three leaflets or cusps: the left coronary cusp LCC; the right coronary cusp RCC; and the non-coronary cusp NCC. As known to those skilled in the art, the right coronary artery includes an ostium or opening RCO in the sinus of Valsalva, superior to the right coronary cusp RCC and inferior to the sinotubular junction (not shown). Similarly, the left coronary artery includes an ostium or opening LCO in the sinus of Valsalva, superior to the left coronary cusp RCC and inferior to the sinotubular junction (not shown). Further, the non-coronary cusp NCC is in the sinus that does not include an ostium or opening for a coronary artery. As known to those skilled in the art, and shown in FIGS. 4A-4B, the leaflets or cusps are joined at commissures. Thus, the left-right commissure LRC is where the left coronary cusp LCC and the right coronary cusp RCC are joined, the right-non-coronary commissure RNC is where the right coronary cusp RCC and the non-coronary cusp NCC are joined, and the left-non-coronary commissure LNC is where the left coronary cusp LCC and the non-coronary cusp NCC are joined. The commissures are not always in the same location for all patients. Therefore, for each commissure location in the idealized drawings of FIGS. 4A-4B, there is a commissure zone, which is a 10-20 degree variation in the location of the commissures. Further, it is noted that the commissures are not exactly 120° apart. Instead, on average, the left-right commissure LRC is closer to the left-non-coronary commissure LNC, at approximately 108°, than to the other two commissures. Further, the location of the ostia or coronary take-off of the left and right coronary arteries may vary approximately 15-20 degrees depending on patient anatomy.

In the idealized alignment of the transcatheter heart valve prosthesis 100 within the native aortic valve, the commissures posts 126A of the stent 102, and hence the commissures of the prosthetic valve 104, will be aligned with the native valve commissures. Thus, the inflow markers 160 will be aligned with the native commissures, as shown in FIG. 4A. Further, since the first outflow marker 170 is disposed on one of the commissure posts 126A, the first outflow marker 170 will also be aligned with one of the native commissures in the idealized native anatomy shown in FIGS. 4A-4B. The second outflow marker 180 is offset from the first outflow marker approximately 60 degrees around the circumference of the stent 102. By “approximately 60 degrees”, it is meant 50 to 70 degrees. In other embodiments, the second outflow marker 180 is circumferentially offset from the first outflow marker 170 by 30 to 90 degrees, or 45 to 75 degrees. Therefore, in the normal view of FIG. 4B, the first outflow marker 170 is shown aligned with the left-right commissure and the second outflow marker 180 is offset approximately 60 degrees around the circumference of the native aortic valve from the left-right commissure, within the left coronary cusp.

The inflow markers 160 and the outflow markers 170, 180 can be used in various viewing angles of imaging systems such as fluoroscopic imaging systems. Imaging systems, such as fluoroscopic imaging systems used during transcatheter aortic valve replacement procedures, generally include a C-arm gantry that enables different viewing angles of the native aortic valve.

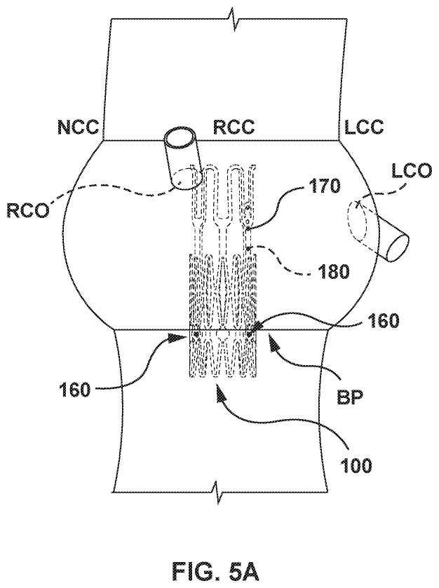

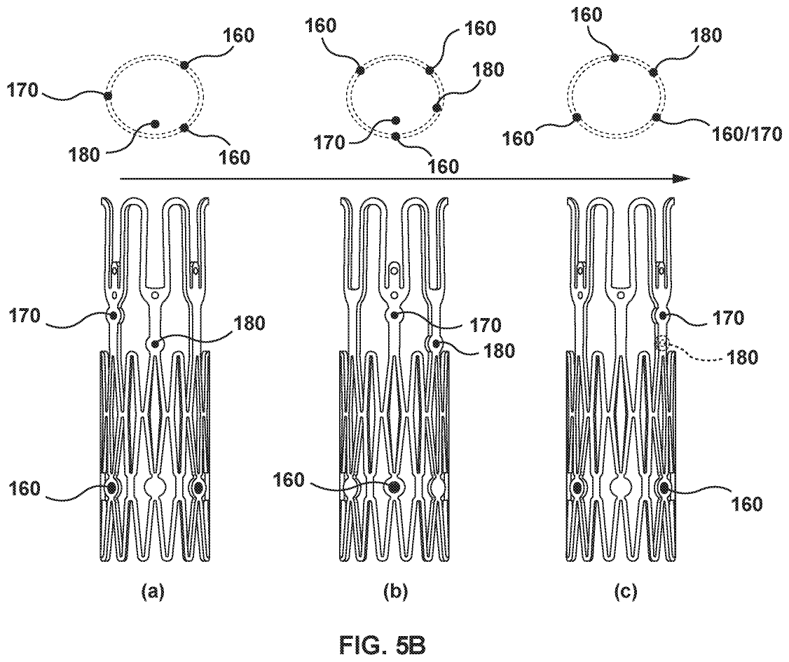

The inflow markers 160 can be utilized to axially align the stent 102 with features in the target site, e.g., basal plane of the right coronary cusp RCC, the left coronary cusp LCC and the non-coronary cusp NCC. The basal plane 340 can be defined as a plane that intersects the nadirs of the right coronary cusp RCC, the left coronary cusp LCC, and the non-coronary cusp NCC. To align the transcatheter heart valve prosthesis 100, the stent 102, via a delivery system can be manipulated (e.g., advanced, retracted, etc.) until the inflow markers 160 align with the basal plane BP, as shown in FIG. 5A. As such, the transcatheter valve prosthesis 100 can be positioned at a proper depth within the target site, thereby ensuring proper engagement with the native tissue. Utilizing three inflow markers 160 facilitates identification of when parallax is present in the transcatheter heart valve prosthesis 100 for a given fluoroscopic viewing angle. If parallax is present in a viewing angle, the inflow markers 160 will not appear in the fluoroscopic image in a line. Changing the viewing angle through operation of the C-arm gantry can be completed to result in the three inflow markers 160 being aligned. Implant depth relative to the native aortic valve cusps can be more accurately assessed with parallax removed.

As noted above, it is also desirable to rotationally align or orient the transcatheter heart valve prosthesis 100, for example, to avoid blocking coronary access. FIGS. 5A-5B illustrate a three-cusp imaging view of a native aortic heart valve with the transcatheter valve prosthesis 100 delivered thereto, but not yet expanded. The three-cusp imaging view is taken such that the non-coronary cusp is centered in the viewing angle, as best seen in FIG. 5A.

In embodiments, the first outflow marker 170 and the second outflow marker 180 can be utilized to circumferentially or rotationally orient the transcatheter heart valve prosthesis 100. That is, the relative appearance and/or location in a 2D image can be utilized to circumferentially or rotationally orient the transcatheter heart valve prosthesis 100. In particular, the relative radial or lateral appearance in 2D image can indicate the relative positioning of the first and second outflow markers 170 and 180 when in a particular image plane or viewing angle. In particular, in the three-cusp view shown in FIG. 5A, the first outflow marker 170 is aligned at the right side of the 2D image, and the second outflow marker 180 is shown axially aligned with the first outflow marker 170. Thus, the first and second outflow markers 170, 180 appear stacked at the right side of the 2D fluoroscopic image, as shown in FIG. 5A, despite the first and second outflow markers 170 and 180 being circumferentially offset from each other by approximately 60 degrees around the circumference of the stent 102, as explained above. The first and second outflow markers 170 and 180 appear stacked in the 2D fluoroscopic image because the second outflow marker 180 is behind the first outflow marker 170. The fluoroscopic image shows the first and second outflow markers 170, 180 due to their radiopacity. Because the relative depth of the first and second outflow markers 170, 180 cannot be seen in a two-dimensional image, they appear stacked.

With the first and second outflow markers 170, 180 stacked at the right side of the transcatheter heart valve prosthesis 100 in the three-cusp viewing angle, the first outflow marker 170 and the commissure post 126A upon which the first outflow marker 170 is disposed are aligned with the left-right commissure LRC, as shown in FIG. 4B. Further, the second outflow marker 180 is disposed generally in the area of the left main coronary artery ostium LCO, as also shown in FIG. 4B. Therefore, the rotational alignment using the first and second outflow markers 170, 180, as described above, enables the commissure posts 126A of the stent 102, and hence the prosthetic valve commissures, to be generally aligned with the native commissures. Such an alignment prevents or reduces the risk of blocking access to the right coronary artery and the left main coronary artery, and provides the benefits of commissural alignment noted above. Further, with the second outflow marker 180 positioned generally at the left main coronary ostium LCO, the second outflow marker 180 can serve as a guide for future procedures that require access to the left main coronary artery.

To align the transcatheter heart valve prosthesis 100, the stent 102 can be rotated, in situ, by a delivery system (not shown) until the first outflow marker 170 and the second outflow marker 180 do not appear radially or laterally offset, i.e., are stacked. FIG. 5B shows several images in the three-cusp viewing angle and corresponding images in the normal view, i.e., as seen from the aorta, as the transcatheter heart valve prosthesis 100 is rotated using a delivery system. For example, in FIG. 5B, panel (a), the first outflow marker 170 and the second outflow marker 180 appear radially or laterally offset. To align the transcatheter valve prosthesis 100, the stent 102 can rotated, in situ, by the delivery system until the first outflow marker 170 and the second outflow marker 180 do not appear radially offset (e.g., are stacked vertically) as illustrated in FIG. 5B, and panel (c).

In embodiments, the first outflow marker 170 and/or the second outflow marker 180, alone, can also be used as a guide to the front or rear location of the first outflow marker 170 appearing in 2D image. The relative motion of the first outflow marker 170 and the second outflow marker 180, when rotated, can be used to indicate the front or rear location of the first outflow marker 170 appearing in the 2D image. In particular, the right or left location of the first outflow marker 170 relative to the second outflow marker 180, during rotation of the stent 102, can indicate the front or rear location. For example, if the second outflow marker 180 is placed on an axial strut 126B to the right of the commissure post 126A containing the first outflow marker 170, the appearance of the second outflow marker 180 to the right of the first outflow marker 170, during rotation, would indicate a front location, as shown in FIG. 5B, panel (a). Likewise, the appearance of the second outflow marker 180 to the left of the first outflow marker 170 would indicate a rear location. The particular movement of the first outflow marker 170 and the second outflow marker 180 may differ based on the location of the markers, the delivery route of the delivery system, and/or the orientation of the transcatheter heart valve prosthesis 100 in the delivery system, for example.

In addition to the inflow markers 160 being used for longitudinal alignment/depth of the transcatheter heart valve prosthesis 100, the inflow markers may also be used for rotational alignment of the transcatheter heart valve prosthesis 100.

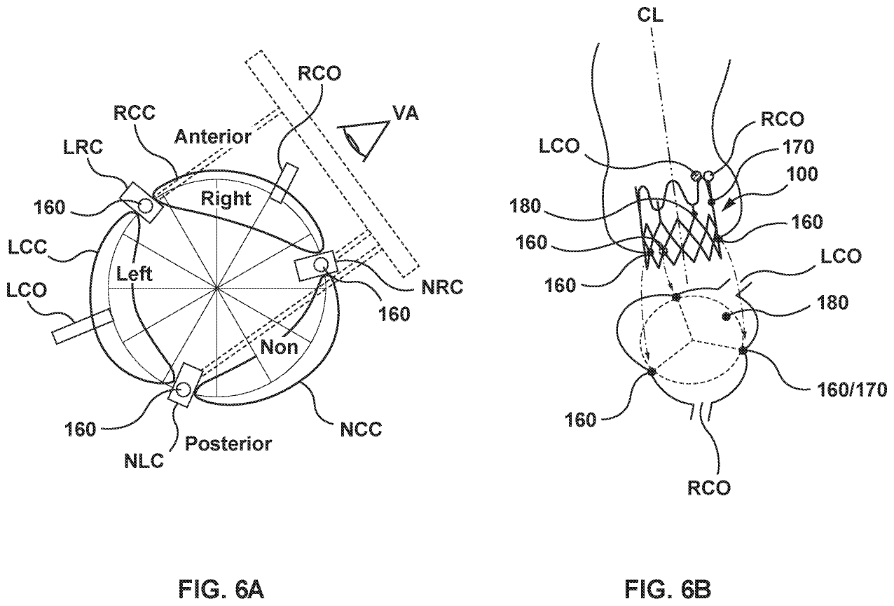

As noted above, imaging systems such as fluoroscopic imaging systems used during transcatheter aortic valve replacement procedures generally include a C-arm gantry that enables different viewing angles of the native aortic valve. One particular viewing angle is a “cusp overlap view” or a “cusp overlap viewing angle”. In the cusp overlap view, as shown in FIG. 6A, the viewing angle VA of the imaging system is such that the right coronary cusp RCC and the left coronary cusp RCC overlap each other. FIG. 6B shows a schematic representation of a fluoroscopic image using the cusp overlap view, also referred to as a “cusp overlap viewing angle image” or “cusp overlap view image”. In FIG. 6B, the right side shows the right coronary cusp RCC and the left coronary cusp LCC aligned with each other, i.e., they overlap. In the cusp overlap view, the non-coronary cusp NCC is to the left of the right coronary cusp RCC and the left coronary cusp LCC. Thus, the left side of FIG. 6B represents the non-coronary cusp NCC. FIG. 6B also shows example locations of the left main coronary ostium LCO and the right coronary ostium RCO, although these would not generally be visible in a fluoroscopic image.

With the above understanding of the cusp overlap view and the inflow markers 160 of the transcatheter heart valve prosthesis 100 as described above, a system and method for rotationally aligning the transcatheter heart valve prosthesis 100 will now be described. As known to those skilled in the art, the transcatheter heart valve prosthesis 100 may be delivered percutaneously via femoral access. In particular, in the example of a balloon-expandable transcatheter heart valve prosthesis, e.g. the transcatheter heart valve prosthesis 100, the prosthesis is radially crimped to a radially compressed configuration and disposed on a balloon of a balloon catheter, for example. Characteristics of a patient's native anatomy may be determined prior to starting the procedure, such as by a CT scan. Using this planning CT, a determination may be made prior to the procedure regarding orientation of the delivery system, and hence the transcatheter heart valve prosthesis, when delivering the transcatheter heart valve prosthesis. For example, and not by way of limitation, a feature of the delivery system may be aligned with a feature of the transcatheter heart valve prosthesis 100 such that there is confidence that the transcatheter heart valve prosthesis 100 will be properly rotationally oriented when delivered to the native aortic valve, i.e., the transcatheter heart valve prosthesis 100 is oriented such as not to block access to the left main coronary artery ostium LCO and the right coronary ostium RCO. Using pre-procedure CT, the orientation of a feature of the delivery system that has a known relationship to a feature of the transcatheter heart valve prosthesis, such as one of the commissures of the transcatheter heart valve prosthesis 100, may be further defined by the specific patient anatomy. Thus, using pre-procedure planning, a prediction can be made regarding a preferred orientation of the delivery system to reduce the risk of coronary artery obstruction and improve/achieve commissural alignment between the prosthetic valve commissures and the native valve commissures. For example, and not by way of limitation, commissural alignment may improve coronary flow and leaflet washout.

Further, during the procedure, the cusp overlap view and inflow markers 160 may be used to confirm that the transcatheter heart valve prosthesis 100 is rotationally aligned such as to not cause coronary obstruction and improve/achieve commissural alignment. The transcatheter heart valve prosthesis 100 may be delivered via a balloon catheter past the native valve leaflets/cusps until the inflow markers 106 are aligned with the annulus of the native heart valve, as explained above. To confirm rotational orientation, the C-arm gantry of the imaging system is located in the cusp overlap view. As shown in FIG. 6B, if two of the inflow markers 160 of the transcatheter heart valve prosthesis 100 can be seen left of the centerline CL and one of the inflow markers 160 can be seen right of the centerline CL, the transcatheter heart valve prosthesis 100 is properly rotationally oriented such as to avoid coronary artery obstruction and improve commissural alignment. In particular, it is desirable for the two inflow markers 160 left of the centerline CL to be substantially aligned. As used herein, “substantially aligned” means circumferentially within one cell or 30 degrees of each other. It is noted that left and right as used regarding FIG. 6B and other fluoroscopy illustrations is with respect to the fluoroscopy image/illustration, not anatomical left and right. FIG. 6B also shows the projection of the inflow markers 160 onto an idealized normal view of the native aortic valve, which would not be part of the fluoroscopic image.

If two of the inflow markers 160 are not seen left of the centerline CL of the fluoroscopy image, and/or two of the inflow markers 160 are seen to the right of the centerline CL of the fluoroscopy image, then the transcatheter heart valve prosthesis 100 may be rotated until there are two inflow markers 160 to the left side of the image in in the cusp overlap view. If the transcatheter heart valve prosthesis 100 needs to be rotated, it may be rotated by rotating a handle of the delivery system. Non-limiting examples of delivery systems that can be rotated at the handle to rotate a distal end of the delivery system are shown and described in U.S. provisional patent application No. 63/129,194, filed Dec. 22, 2020, the contents of which are incorporated by reference herein in their entirety, or any other suitable delivery system.

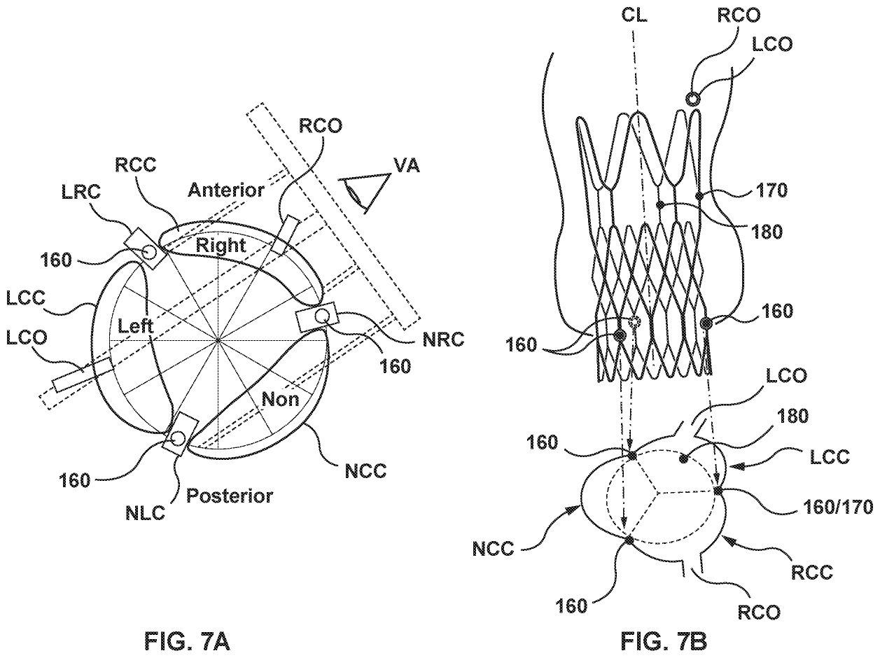

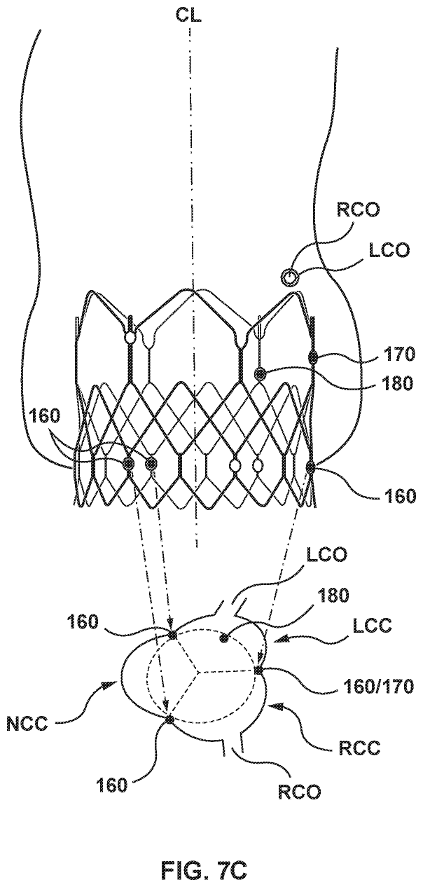

Another viewing angle is referred to herein as the “coronary overlap view” or “coronary overlap viewing angle”. In the coronary overlap view, during the pre-procedure CT work-up, the location of the coronary ostia (i.e., the openings of the coronary arteries into the sinus of the native aortic valve) are located. Using these locations, it can be determined the proper angle of the C-arm of the imaging system such that the coronary ostia overlap. For example, FIG. 7A shows an example idealized native aortic valve (e.g. the native commissures are 120 degrees apart), with the location of the left coronary ostium LCO and right coronary ostium RCO marked. As shown in FIG. 7A, the viewing angle VA of the imaging system is selected such that the right coronary artery ostium RCO and the left main coronary artery ostium LCO overlap each other. As noted above, patient anatomies vary from this idealized representation. FIG. 7B shows a schematic representation of a fluoroscopic image with the viewing angle set for the coronary overlap view, referred to as the “coronary overlap viewing angle image” or the “coronary overlap vie image”. It is noted that the coronary arteries are not generally visible in the fluoroscopic image, but by using the coronary overlap view, it is known where the coronary ostia are located. Further, because the coronary ostia are aligned in the coronary overlap view, they are commonly located in the 2-Dimensional view of the fluoroscopic image (i.e., one is behind the other). Therefore, knowing the location of the coronary ostia, a clinician can check the location of the commissures of the valve structure of the transcatheter heart valve prosthesis 100 to ensure that none of the prosthetic commissures are aligned or in near alignment with the coronary ostia. In particular, with the transcatheter heart valve prosthesis 100, the commissure posts 126A are aligned with the prosthetic commissures, and the inflow markers 160 are aligned with the commissure posts 126A. Therefore, the coronary overlap view and the inflow markers 160 can be used to avoid coronary obstruction and improve/achieve commissural alignment.

As explained above, the planning CT can be used to make a determination prior to the procedure to best orient the delivery system such that rotation of the delivery system at the target site may not be necessary. Further, during the procedure, the coronary overlap view and the inflow markers 160 may be used to confirm that the transcatheter heart valve prosthesis is rotationally aligned such as to not cause coronary obstruction and provide commissural alignment. As explained above, the delivery system is advanced past the native valve leaflets/cusps until the inflow markers 160 are aligned with the annulus of the native heart valve for depth alignment.

For rotational alignment, with the imaging system in the coronary overlap view, if two inflow markers 160 are shown left of the center-line CL of the image and one of the inflow markers 160 is shown to the right of the center-line CL of the image, as shown in FIG. 7B, then the transcatheter heart valve prosthesis 100 is properly rotationally aligned such as to avoid coronary blockage and improve/achieve commissural alignment. In particular, it is desirable for the two inflow markers 160 left of the centerline CL to be substantially aligned. As used herein, “substantially aligned” means within circumferentially within one cell or 30 degrees of each other.

FIG. 7C shows the transcatheter heart valve prosthesis 100 shown in FIG. 7B after it has been radially expanded. As can be seen in FIG. 7C, two of the inflow markers 160 are to the left of the centerline CL and one of the inflow markers 160 is to the extreme right of the image. This arrangement shows the proper rotational alignment to avoid coronary obstruction and improve/achieve commissural alignment.

It is also noted that using the planning CT and the coronary overlap view, the location of the coronary artery ostia can be marked on the imaging screen. In such an example, viewing the inflow markers 160 in the coronary overlap view as compared to the marked location of coronary artery ostia can confirm proper rotational orientation to avoid coronary artery obstruction and improve/achieve commissural alignment.

If two of the inflow markers 160 are not seen left of the centerline CL of the fluoroscopy image, and/or two of the inflow markers 160 are seen to the right of the centerline CL of the fluoroscopy image, and/or an inflow marker 160 is seen aligned with the marked location of the coronary artery ostia, then the transcatheter heart valve prosthesis 100 may be rotated until there are two inflow markers 160 to the left side of the image in in the coronary overlap view. If the transcatheter heart valve prosthesis 100 needs to be rotated, it may be rotated by rotating a handle of the delivery system. Non-limiting examples of delivery systems that can be rotated at the handle to rotate a distal end of the delivery system are shown and described in U.S. provisional patent application No. 63/129,194, filed Dec. 22, 2020, the contents of which are incorporated by reference herein in their entirety, or any other suitable delivery system.

The outflow markers 170, 180 can also be used in the coronary overlap view and/or the cusp overlap view to mark the location of the left main coronary artery ostium LCO. As described above, FIG. 7B shows a schematic representation of a fluoroscopic image with the viewing angle set for the coronary overlap view. FIG. 7C shows the same view with the transcatheter heart valve prosthesis 100 in the radially expanded deployed configuration. In addition to using the inflow markers 160 for rotational alignment, the outflow markers 170, 180 may also be used to mark the location of the left main coronary artery ostium LCO. In particular, as shown in FIG. 7B, the first outflow marker 170 is axially aligned with the inflow marker 160 to the right of the centerline in the coronary overlap view image. The second outflow marker 180 is shown in FIGS. 7B and 7C to the left of the first outflow marker 170. Due to the location of the second outflow marker 180 relative to the first outflow marker 170, it is known that in the images of FIG. 7B and 7C the second outflow marker 180 is located in the rear portion of the transcatheter heart valve prosthesis 100 and the rear portion of the native heart valve relative to the viewing angle. In other words, although the images in FIGS. 7B and 7C are two-dimensional, it is known that portions of the tubular transcatheter heart valve prosthesis 100 are towards the front of the native heart valve in the image and portions of the tubular transcatheter heart valve prosthesis 100 are towards the rear of the native heart valve in the image. The location of the first outflow marker 170 and the location of the second outflow marker 180 relative to the first outflow marker 170 indicate that in the coronary overlap viewing angle image, the second outflow marker 180 is towards the rear of the image. As projected onto the normal views also shown in FIGS. 7B and 7C, this rotational orientation results in the second outflow marker 180 being located at or near the left main coronary artery ostium LCO, as shown in FIGS. 7B and 7C. Thus, the second outflow marker 180 can serve as a guide for future procedures that require access to the left main coronary artery. In other words, if after implantation of the transcatheter heart valve prosthesis 100, another procedure is required, such as, but not limited to balloon angioplasty or stenting of a coronary artery, the location of the left main coronary artery ostium LCO will be marked by the second outflow marker 180 to assist in guiding a delivery device to the left main coronary artery ostium LCO. Those skilled in the art will recognize that the marking of the left main coronary artery ostium LCO in the cusp overlap view will work the same as described above with respect to the coronary overlap view. Thus, the description of using the second outflow marker 180 to mark the left main coronary artery ostium is incorporated into the description of the cusp overlap view.

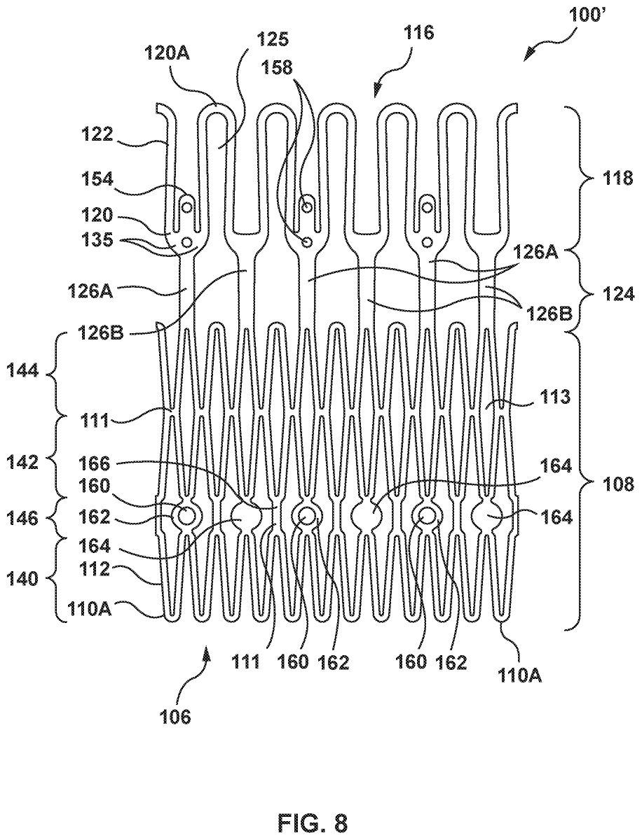

The transcatheter heart valve prosthesis 100 described above includes three inflow markers 160 and two outflow markers 170, 180. In an alternative embodiment, a transcatheter heart valve prosthesis 100′ may include only the three inflow markers 160, and no outflow markers 170, 180. FIG. 8 is a laid open, flat illustration of such the transcatheter heart valve prosthesis 100′. All features of the transcatheter heart valve prosthesis 100 are incorporated into the description of the transcatheter heart valve prosthesis 100′. Further, all methods described above with respect to the transcatheter heart valve prosthesis 100 are incorporated into the description of the transcatheter heart valve prosthesis 100′, except for use of the outflow markers 170, 180 of the transcatheter heart valve prosthesis 100 for rotational alignment in the three-cusp view and for use in marking the left main coronary ostium LCO in the three-cusp, cusp overlap, or coronary overlap views, as the transcatheter heart valve prosthesis 100′ does not include such outflow markers.

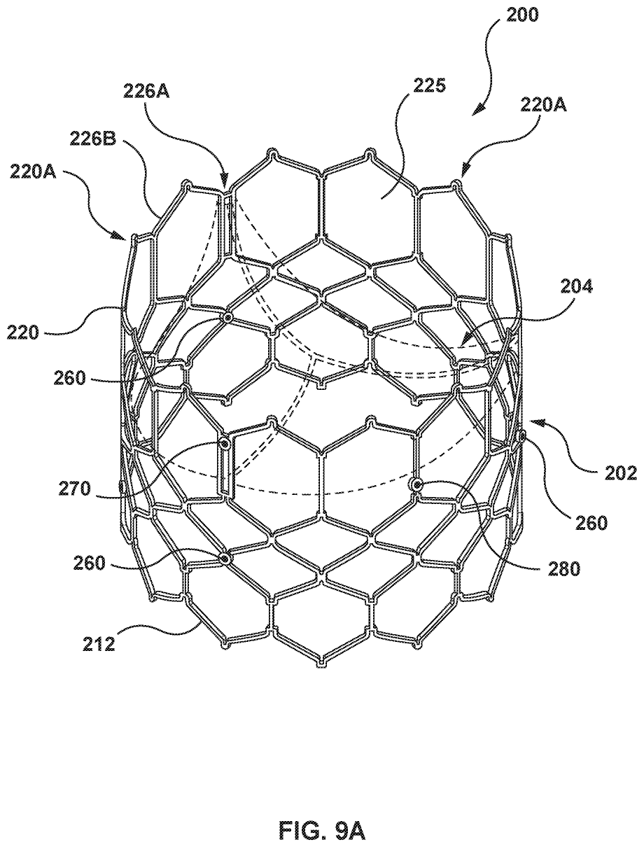

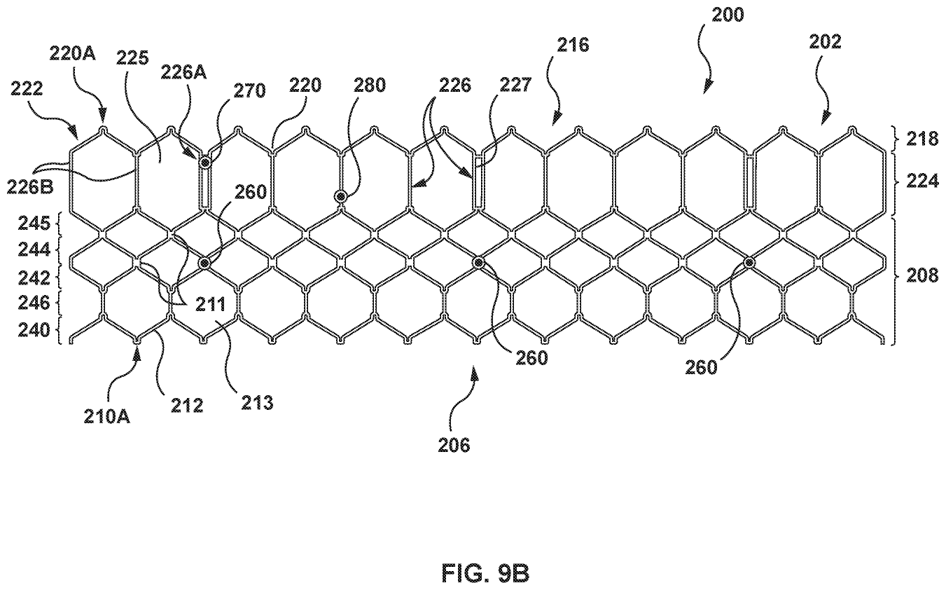

FIGS. 9A-9B illustrate a transcatheter heart valve prosthesis 200 in accordance with another embodiment hereof. The transcatheter heart valve prosthesis 200 includes a radially-expandable stent 202 and a prosthetic valve 204 coupled within the stent 202. The prosthetic valve 204 is shown in dashed lines for clarity. The transcatheter heart valve prosthesis 200 further includes three inflow markers 260 and two outflow markers 270, 280, as described in more detail below. In embodiments, the inflow markers 260 and the outflow markers 270, 280 can be utilized in orientation (e.g., axial alignment, tilt alignment, circumferential (rotational) alignment, etc.) of the transcatheter heart valve prosthesis 200, in situ, as discussed in detail below.

One skilled in the art will realize that FIGS. 9A and 9B illustrate one example of an implantable medical device and that existing components illustrated in FIGS. 9A and 9B may be removed and/or additional components may be added. Additionally, while the transcatheter heart valve prosthesis 200 is described below as including three inflow markers 260 and two outflow markers 270, 280, one skilled in the art will realize that the transcatheter heart valve prosthesis 200 can include additional markers, for example, any of the markers described herein. Moreover, while examples of operations and advantages of transcatheter heart valve prosthesis 200 including the inflow markers 260 and the outflow markers 270, 280 are discussed below, one skilled in the art will realize any of the operations and processes described herein can be performed using the transcatheter heart valve prosthesis 200.

The stent 202 has an expanded configuration, which is shown FIG. 9A. The stent 202 can be made from a plastically deformable material such that, when expanded, the stent 202 maintains its radially expanded configuration. The stent 202 can be formed from any of various suitable plastically-expandable materials (e.g., stainless steel, etc.) or self-expanding materials (e.g., Nitinol) as known in the art. For example, the stent 202 can be formed from stainless steel or other suitable metal, such as platinum iridium, cobalt chromium alloys such as MP35N, or various types of polymers or other materials known to those skilled in the art, including said materials coated with various surface deposits to improve clinical functionality. In some embodiments, the stent 202 can be self-expanding.

The stent 202 can be formed from a unitary frame or scaffold having an inflow portion 208, an outflow portion 218, and a transition portion 224 bridging, connecting, or otherwise extending between the inflow portion 208 and the outflow portion 218. The stent 202 can be a generally tubular component defining a central lumen or passageway and can have an inflow or proximal end 206 and an outflow or distal end 216. In some embodiments, when expanded, a diameter of the inflow end 206 of the stent 202 can be the same as a diameter of the outflow end 216 of the stent 202. The stent 202 can be formed by a laser-cut manufacturing method and/or another conventional stent forming method as would be understood by one of ordinary skill in the art. The cross-section of the stent 202 can be trapezoidal, circular, ellipsoidal, rectangular, hexagonal, square, or other polygonal shape, although at present it is believed that trapezoidal, circular or ellipsoidal may be preferable when utilized with the replacement of an aortic valve. FIG. 9B shows an open, flat view of an example of the stent 202.

The inflow portion 208 can be formed proximate to the inflow end 206 of the stent 202. The inflow portion 208 of the stent 202 may be formed with rows of struts 212 and crowns or bends 210 coupling circumferentially adjacent struts to each other. The struts 212 may also be referred to as angled struts or inflow struts or angled inflow struts. The crowns 210 may also be referred to as inflow crowns. Nodes 211 are formed at intersections of a crowns 210 of adjacent rows of struts and crowns. In some embodiments, the inflow end 206 of the stent 202 can include a total of twelve endmost inflow crowns 210A. Cells 213 of the inflow portion of the stent 202 are defined by a pair of circumferentially adjacent struts 212 of a row of struts and crowns, a pair of circumferentially adjacent struts 212 of an adjacent row of struts and crowns, and nodes 211 between the adjacent rows of struts and crowns. Cells 213 define open spaces in the inflow portion 208 of the stent 202.

The outflow portion 218 can be formed proximate to the outflow end 216 of the stent 202. The outflow portion 218 can be configured in a shape that forms a central lumen or passageway, for example, a ring. The outflow portion 218 can include a plurality of crowns 220 and a plurality of struts 222 with each crown 220 being formed between a pair of opposing struts 222. Each crown 220 can be a curved segment or bend coupling opposing struts 222. The struts 222 may also be referred to as outflow struts or angled struts or angled outflow struts. The crowns 220 may also be referred to as outflow crowns. A series of endmost outflow crowns 220A are formed at the outflow end 216 of the stent 202. In some embodiments, the outflow end 216 of the stent 202 can have a total of twelve endmost outflow crowns 220A. In the embodiment shown, the outflow portion 218 includes a single row of struts 212 and crowns 220. In other embodiments, additional rows of struts and crowns may be included in the outflow portion 218.

The transition portion 224 bridges, connects, or otherwise extends between the inflow portion 208 and the outflow portion 218. In some embodiments, the transition portion 224 can include a minimum of three axial frame members 226, each axial frame member 226 extending between an outflow crown 220 of the outflow portion 218 and a crown 210 of the inflow portion 208. Each axial frame member 226 can be connected to a crown 220 of the outflow portion 218 and connected to a crown 210 of the inflow portion 208. The axial frame members 226 can be substantially parallel to the central longitudinal axis of the stent 202. By “substantially parallel” it is meant that the axial frame members 226 may be within 10 degrees of parallel to the central longitudinal axis. Each axial frame member 226 can be disposed circumferentially approximately halfway between a pair of adjacent endmost outflow crowns 220A. In embodiments, each axial frame member 226 may terminate at a distal-most crown 210 of the inflow section 208 and a proximal-most crown 220 of the outflow section 218. In other embodiments, each axial frame member 226 may terminate at the distal-most row of struts and crowns of the inflow portion 208 and the proximal-most row of struts and crowns of the outflow portion 218. While the stent 224 has been described as including a transition portion 224, one skilled in the art will realize that the transition portion 224 may form a portion of the inflow portion 208 and/or the outflow portion 218

In an embodiment, the transition portion 224 can include up to twelve axial frame members 226, with three of the axial frame members 226 being commissure posts 226A and nine of the axial frame members 226 being axial struts 226B. The commissure posts 226A and the axial struts 226B can be being alternatingly positioned, as illustrated, for example, in FIG. 9B with three axial struts 226B being positioned between two adjacent commissure posts 226A. The commissure posts 226A can be circumferentially spaced apart and aligned with and attached to a respective commissure of the three leaflets of the prosthetic valve 204, and the three axial struts 226B can be disposed between adjacent commissure posts 226A. The axial frame members 226 may aid in valve alignment and coaptation. More particularly, the axial frame members 226 reinforce or strengthen the commissure region of the prosthetic valve by shaping the leaflets and supporting the leaflets during opening and closing thereof, and thus provide more reliable leaflet coaptation. In addition, the axial frame members 226 maximize symmetrical cell expansion.

In embodiments, each of the commissure posts 226A includes a commissure window 227. Each commissure post 226A mounts a respective commissure of the leaflet structure of a replacement valve. In embodiments, at each commissure post 226A, respective ends of two leaflets extend through the commissure window 227 and are secured to the commissure post 226A.

In an embodiment, with a single row of struts 222 and crowns 220 in the outflow portion 218, the endmost outflow crowns 220A are not connected to the axial frame members 226 but rather may be considered to be free or unattached while the remaining outflow crowns 220 of the single row of the outflow portion 218 are connected to the axial frame members 226 and disposed closer to the inflow end 206 than the endmost outflow crowns 220A are to the inflow end 206. In the embodiment shown, the stent 202 includes a single row of struts 222 and crowns 220 coupled to the axial frame members 226 and defining the outflow end 216 of the stent 202. Further, in the embodiment shown, exactly two struts 222 and a single crown 220 of the outflow portion 118 are disposed circumferentially between adjacent axial frame members 226. Such an arrangement can provide a series of twelve endmost cells 225 formed at the outflow portion 218 of the stent 202.

In embodiments, each endmost cell 225 can define an open space in the stent 202, which is formed in any type of shape, in the radially expanded configuration, for example, as shown in FIG. 9A. Each endmost cell 225 can be defined by two circumferentially adjacent struts 222 of the outflow portion 218, the endmost crown 220A connecting the struts 222, two circumferentially adjacent struts 212 of the inflow portion 208, the crowns 210 connecting the struts 212, and two circumferentially adjacent axial frame members 226 of the transition portion 224. The endmost cells 225 of the outflow portion 218 can be relatively larger than the cells 213 of the inflow portion 208 to improve access to the coronary arteries when used for aortic valve replacement. More particularly, the endmost cells 225 of the outflow portion 218 can be configured to be of sufficient size to be easily crossed with a coronary guide catheter into either the right coronary artery or the left main coronary artery once the transcatheter valve prosthesis 200 is deployed, in situ.

In an embodiment, the inflow portion 208 can include exactly four rows of struts 212 and crowns 210 between the inflow end of the axial frame members 226 and the inflow end 206 of the stent 202. The rows struts and crowns may also be referred to as rows of angled struts. In particular, as illustrated in FIG. 9B, the stent 202 can include a first row 240 of angled struts 212 formed proximate to the inflow end 206, a second row 242 of angled struts 212 formed longitudinally adjacent the first row 240 and towards the outflow end 216 of the stent 202 relative to the first row 240, a third row 244 of angled struts 212 formed longitudinally adjacent the second row 242 and towards the outflow end 216 of the stent 202 relative to the second row 242, and a fourth row 245 of angled struts 212 formed longitudinally adjacent the third row 244 and towards the outflow end 216 of the stent 202 relative to the third row 242. The inflow portion 208 further includes an elongated junction 246 between the first row 240 and the second row 242. As can be seen in FIG. 9B, the junction 246 is longitudinally elongated relative the nodes 211 formed between the second row 242 and the third row 244, and the nodes 211 formed between the third row 244 and the fourth row 245. The elongated junction 246 may also be referred to as a row of axial struts connecting the first row 240 and the second row 242.

In embodiments, to ensure the proper placement in the native anatomy of a subject, the transcatheter valve prosthesis 200 can include the inflow markers 260 and the outflow markers 270, 280, as described in more detail below. The inflow markers 260 may be used for depth (longitudinal) alignment and/or rotational alignment of the stent 202, as described in more detail below. The outflow markers 270, 280 may be used for rotational alignment and can operate as a guide for determining a front or rear location the outflow markers 270, 280 in 2D images during implantation, as described below.

In an embodiment, as shown in FIGS. 9A-9B, the first outflow marker 270 can be positioned on a commissure post 226A and the second outflow marker 280 can be positioned on an axial strut 226B between the commissure post 226A on which the first outflow marker 270 is positioned and an adjacent commissure post 226A. In the embodiment of FIGS. 9A-9B the second outflow marker 280 is positioned on the second axial strut 226B in a counter-clockwise direction from the commissure post 226A upon which the first outflow marker 270 is positioned, when the stent 202 is viewed from its outer surface. Further, as shown in FIGS. 9A-9B, the second outflow marker 280 can be positioned closer to the inflow end 206 of the stent 202 than the first outflow marker 270 is to the inflow end 206 of the stent 202. In other words, the second outflow marker 280 is positional proximally or upstream relative to the first outflow marker 270. Stated another way, the first outflow marker 270 is positioned adjacent the outflow end of a commissure post 226A and the second outflow marker 280 is positioned adjacent the inflow end of an axial strut 226B. In other words, the first and second outflow markers 270, 280 are axially offset. In other embodiments, the longitudinal orientation of the first and second outflow markers 270, 280 may be reversed such that the first outflow marker 270 is proximal to/upstream of the second outflow marker 280.

In embodiments, the outflow markers 270, 280 can be attached to the stent 202 within containment members formed in a commissure post 226A and an axial strut 226B, respectively, as described above with respect to the transcatheter heart valve prosthesis 100. Details of containment members are provided above with respect to the transcatheter heart valve prosthesis 100 and in U.S. patent application Ser. No. 17/187,261, filed Feb. 26, 2021, both of which are incorporated by reference into the present embodiment herein in their entirety. In other embodiments, the outflow markers 270, 280 need not be located within containment members. For example, and not by way of limitation, the outflow markers 270, 280 may comprise radiopaque bands that are attached to the commissure post 226A and the axial strut 226B, respectively. In another example, and not by way of limitation, the outflow markers 270, 280 may be formed by applying radiopaque materials to the commissure post 226A and the axial strut 226B, respectively, in any shape. One skilled in the art will realize that the outflow markers 270, 280 may be attached to or formed on the stent 202 utilizing any processes as required by the design of the stent 202 and/or application of the transcatheter valve prosthesis 200.

In any embodiment, the first and second outflow markers 270, 280 can be formed to dimensions such that the first and second outflow markers 270, 280 do not adversely affect the operation of the transcatheter valve prosthesis 200. For example, the first and second outflow markers 270 can be formed to not extend beyond the exterior diameter of the stent 202 or extend into the central lumen of the stent 202. In other words, it is preferable for the first outflow marker 270 to have a radial depth that is equal to or less than the radial depth of the commissure post 226A and for the second outflow marker 280 to have a radial depth that is equal to or less than the radial depth of the axial strut 226B. The first and second outflow markers 270, 280 (and any containment member) may be shaped and dimensioned as described above with respect to the first and second outflow markers 270, 280 of the transcatheter heart valve prosthesis 100, and that description and variations thereof is incorporated into and applicable to the first and second outflow markers 270, 280.

In the embodiment of FIGS. 9A-9B, the transcatheter heart valve prosthesis 200 includes three inflow markers 260. The inflow markers 260 are each positioned towards the inflow end 206 of the stent 202 in the inflow portion 208. In an embodiment, as illustrated in FIGS. 9A-9B, the inflow markers 260 are positioned between the second row 242 of angled struts and the third row 144 of angled struts in the inflow portion 208, at the nodes 211. As also shown in FIGS. 9A-9B, the inflow markers 260 are axially aligned with the commissure posts 226A. In other words, each inflow marker 260 is located along a common longitudinal axis with one of the commissure posts 226A. Stated another way, each inflow marker 260 is located “below” one of the commissure posts 226A. As used herein for the inflow markers 260, the term “axially aligned” means that each inflow marker is within one cell of the corresponding commissure post 226A.

In embodiments, each inflow marker 260 can be attached to the stent 202 within a respective containment member. Details of containment members may be found above and in U.S. patent application Ser. No. 17/187,261, filed Feb. 26, 2021, which is incorporated by reference herein in its entirety. In other embodiments, the inflow markers 260 need not be located within containment members. For example, and not by way of limitation, the inflow markers 260 may comprise radiopaque bands that are attached to the inflow portion 208. In another example, and not by way of limitation, the inflow markers 260 may be formed by applying radiopaque materials to the inflow portion 208 in any shape. One skilled in the art will realize that the inflow markers 260 may be attached to or formed on the stent 202 utilizing any processes as required by the design of the stent 202 and/or application of the transcatheter heart valve prosthesis 200.

In any embodiment, the inflow markers 260 can be formed to dimensions such that the inflow markers 260 do not adversely affect the operation of the transcatheter valve prosthesis 200. For example, the inflow markers 260 can be formed to not extend beyond the exterior diameter of the stent 202 or extend into the central lumen of the stent 202. In other words, it is preferable for the inflow markers 260 to have a radial depth that is equal to or less than the radial depth of stent 202 where the outflow markers 260 are coupled to the stent 202, such as the nodes 211 or the struts 212. The inflow markers 260 (and any containment member) may be shaped and dimensioned as described above with respect to the inflow markers 160 of the transcatheter heart valve prosthesis 100, and that description and variations thereof is incorporated into and applicable to the inflow markers 260 of the transcatheter heart valve prosthesis 200.

In any embodiment, the inflow markers 260 and/or the outflow markers 270, 280 include radiopaque or other material that allow the inflow markers 260 and the outflow markers 270, 280 to be detected and/or viewed via fluoroscopy during the installation of the transcatheter valve prosthesis 200. Examples of radiopaque materials include metals, e.g., stainless steel, titanium, tungsten, tantalum, gold, platinum, platinum-iridium, and/or other polymeric materials, e.g., nylon, polyurethane, silicone, PEBAX, PET, polyethylene, that have been mixed or compounded with compounds of barium, bismuth and/or zirconium, e.g., barium sulfate, zirconium oxide, bismuth sub-carbonate, etc.

In embodiments, the inflow markers 260 and the outflow markers 270, 280 can be utilized to orient (e.g., axial alignment, tilt alignment, circumferential (rotational) alignment, etc.) the transcatheter valve prosthesis 100, in situ, during installation as described below. In particular, as noted briefly above, when installing the transcatheter heart valve prosthesis 200, it is desirable to properly align the stent 202 within the target site. For example, the transcatheter heart valve prosthesis 200 needs to be properly aligned axially, so that the transcatheter valve prosthesis 200 properly engages the native leaflets/tissue of the target site, e.g., the aortic annulus, without causing conduction blockages by implanting too deep or causing an embolization of the transcatheter heart valve prosthesis 200 by implanting too high. Likewise, the transcatheter valve prosthesis 200 needs to be aligned circumferentially or rotationally. When being positioned, in situ, it is very important to avoid blocking the right coronary artery ostium RCO and/or the left main coronary artery ostium LCO. Proper circumferential or rotational orientation within the target site reduces the risk of blocking coronary access. Further, proper rotational alignment provides alignment or near alignment of the prosthetic valve commissures with the native valve commissures. Commissural alignment may improve, for example, coronary flow and leaflet washout.

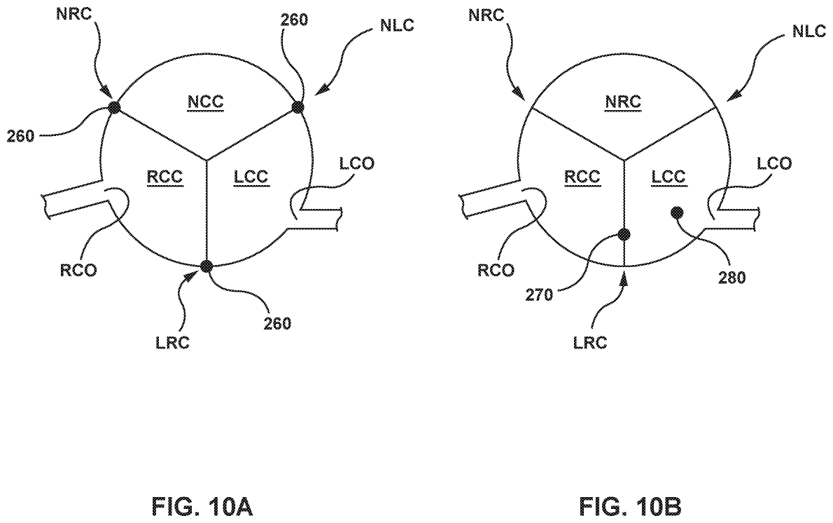

FIGS. 10A-10B show an example of an idealized native aortic valve (e.g. the native commissures are 120 degrees apart) as would be viewed from the aorta, also referred to as the normal or perpendicular view. As shown in FIGS. 10A-10B, the native aortic valve includes three leaflets or cusps: the left coronary cusp LCC; the right coronary cusp RCC; and the non-coronary cusp NCC. As known to those skilled in the art, the right coronary artery includes an ostium or opening RCO in the sinus of Valsalva, superior to the right coronary cusp RCC and inferior to the sinotubular junction (not shown). Similarly, the left coronary artery includes an ostium or opening LCO in the sinus of Valsalva, superior to the left coronary cusp RCC and inferior to the sinotubular junction (not shown). Further, the non-coronary cusp NCC is in the sinus that does not include an ostium or opening for a coronary artery. As known to those skilled in the art, and shown in FIGS. 10A-10B, the leaflets or cusps are joined at commissures. Thus, the left-right commissure LRC is where the left coronary cusp LCC and the right coronary cusp RCC are joined, the right-non-coronary commissure RNC is where the right coronary cusp RCC and the non-coronary cusp NCC are joined, and the left-non-coronary commissure LNC is where the left coronary cusp LCC and the non-coronary cusp NCC are joined. The commissures are not always in the same location for all patients. Therefore, for each commissure location in the idealized drawings of FIGS. 10A-10B, there is a commissure zone, which is a 10-20 degree variation in the location of the commissures. Further, it is noted that the commissures are not exactly 120 degrees apart. Instead, on average, the left-right commissure LRC is closer to the left-non-coronary commissure LNC, at approximately 108°, than to the other two commissures. Further, the location of the ostia or coronary take-off of the left and right coronary arteries may vary approximately 15-20 degrees depending on patient anatomy.

In the idealized alignment of the transcatheter heart valve prosthesis 200 within the native aortic valve, the commissures posts 226A of the stent 202, and hence the commissures of the prosthetic valve 204, will be aligned with the native valve commissures. Thus, the inflow markers 260 will be aligned with the native commissures, as shown in FIG. 10A. Further, since the first outflow marker 270 is disposed on one of the commissure posts 226A, the first outflow marker 270 will also be aligned with one of the native commissures in the idealized native anatomy, as shown in FIG. 10B. The second outflow marker 280 is offset from the first outflow marker approximately 60 degrees around the circumference of the stent 202. By “approximately 60 degrees”, it is meant 50 to 70 degrees. In other embodiments, the second outflow marker 280 is circumferentially offset from the first outflow marker 270 by 30 to 90 degrees, or 45 to 75 degrees. Therefore, in the normal view of FIG. 10B, the first outflow marker 270 is shown aligned with the left-right commissure LRC and the second outflow marker 280 is offset approximately 60 degrees around the circumference of the native aortic valve from the left-right commissure LRC, within the left coronary cusp LCC, which also places the second outflow marker 280 adjacent the left main coronary artery ostium LCO.

The inflow markers 260 and the outflow markers 270, 280 can be used in various viewing angles of imaging systems such as fluoroscopic imaging systems. Imaging systems, such as fluoroscopic imaging systems used during transcatheter aortic valve replacement procedures, generally include a C-arm gantry that enables different viewing angles of the native aortic valve.

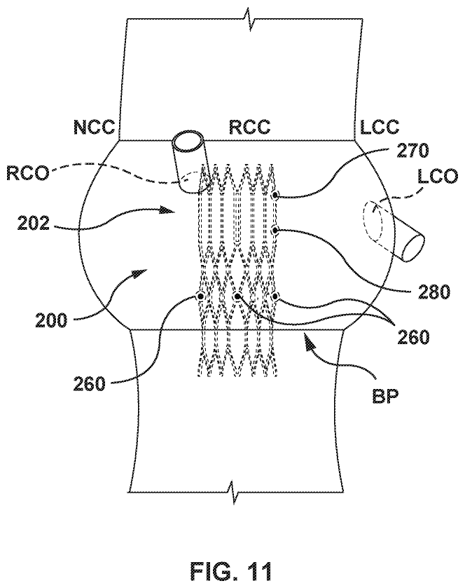

The inflow markers 260 can be utilized to axially align the stent 202 with features in the target site, e.g., basal plane of the right coronary cusp RCC, the left coronary cusp LCC and the non-coronary cusp NCC. The basal plane BP can be defined as a plane that intersects the nadirs of the right coronary cusp RCC, the left coronary cusp LCC, and the non-coronary cusp NCC. To align the transcatheter heart valve prosthesis 200, the stent 202, via a delivery system can be manipulated (e.g., advanced, retracted, etc.) until the inflow markers 260 align near the basal plane BP, as shown in FIG. 11A. As such, the transcatheter valve prosthesis 200 can be positioned at a proper depth within the target site, thereby ensuring proper engagement with the native tissue. Utilizing three inflow markers 260 facilitates identification of when parallax is present in the transcatheter heart valve prosthesis 200 for a given fluoroscopic viewing angle. If parallax is present in a viewing angle, the inflow markers 260 will not appear in the fluoroscopic image in a line. Changing the viewing angle through operation of the C-arm gantry can be completed to result in the three inflow markers 260 being aligned. Implant depth relative to the native aortic valve cusps can be more accurately assessed with parallax removed.

As noted above, it is also desirable to rotationally align or orient the transcatheter heart valve prosthesis 200, for example, to avoid blocking coronary access. FIG. 11 illustrates a three-cusp imaging view of a native aortic heart valve with the transcatheter valve prosthesis 200 delivered thereto, but not yet expanded. The three-cusp imaging view is taken such that the non-coronary cusp is centered in the viewing angle, as best seen in FIG. 11.

In embodiments, the first outflow marker 270 and the second outflow marker 280 can be utilized to align circumferential or rotational orientation of the transcatheter heart valve prosthesis 200. That is, the relative appearance and/or location in a 2D image can be utilized to circumferentially or rotationally orient the transcatheter heart valve prosthesis 200. In particular, the relative radial appearance in 2D image can indicate the relative positioning of the first and second outflow markers 270 and 280 when in a particular image plane or viewing angle. In particular, in the three-cusp view shown in FIG. 11, the first outflow marker 270 is aligned at the right side of the 2D image, and the second outflow marker 280 is shown axially aligned with the first outflow marker 270. Thus, the first and second outflow markers 270, 280 appear stacked at the right side of the 2D fluoroscopic image, as shown in FIG. 11, despite the first and second outflow markers 270 and 280 being circumferentially offset from each other by approximately 60 degrees around the circumference of the stent 202, as explained above. The first and second outflow markers 270 and 280 appear stacked in the 2D fluoroscopic image because the second outflow marker 280 is behind the first outflow marker 270. The fluoroscopic image shows the first and second outflow markers 270, 280 due to their radiopacity. Because the relative depth of the first and second outflow markers 270, 280 cannot be seen in a two-dimensional image, they appear stacked.

With the first and second outflow markers 270, 280 stacked at the right side of the transcatheter heart valve prosthesis 200 in the three-cusp viewing angle, the first outflow marker 270 and the commissure post 226A upon which the first outflow marker 270 is disposed are aligned with the left-right commissure LRC, as shown in FIG. 10B. Further, the second outflow marker 280 is disposed generally in the area of the left main coronary artery ostium LCO, as also shown in FIG. 10B. Therefore, the rotational alignment using the first and second outflow markers 270, 280, as described above, enables the commissure posts 226A of the stent 202, and hence the prosthetic valve commissures, to be generally aligned with the native valve commissures. Such an alignment prevents or reduces the risk of blocking access to the right coronary artery and the left main coronary artery and provides the benefits of commissural alignment discussed above. Further, with the second outflow marker 280 positioned generally at the left main coronary ostium LCO, the second outflow marker 280 can serve as a guide for future procedures that require access to the left main coronary artery.

To align the transcatheter heart valve prosthesis 200, the stent 202 can be rotated, in situ, by a delivery system (not shown) until the first outflow marker 270 and the second outflow marker 280 do not appear radially offset, i.e., are stacked. The rotational alignment and rotation of the delivery system can be accomplished as explained above with respect to FIG. 5B. The method applies equally to the embodiment of FIG. 11. Further, as also explained with respect to FIG. 5B, and applying equally to the embodiment of FIG. 11, the first outflow marker 270 and/or the second outflow marker 280, can also be used as a guide to the front or rear location of the first outflow marker 270 appearing in 2D image.

In addition to the inflow markers 260 being used for longitudinal alignment/depth of the transcatheter heart valve prosthesis 200, the inflow markers may also be used for rotational alignment of the transcatheter heart valve prosthesis 200.

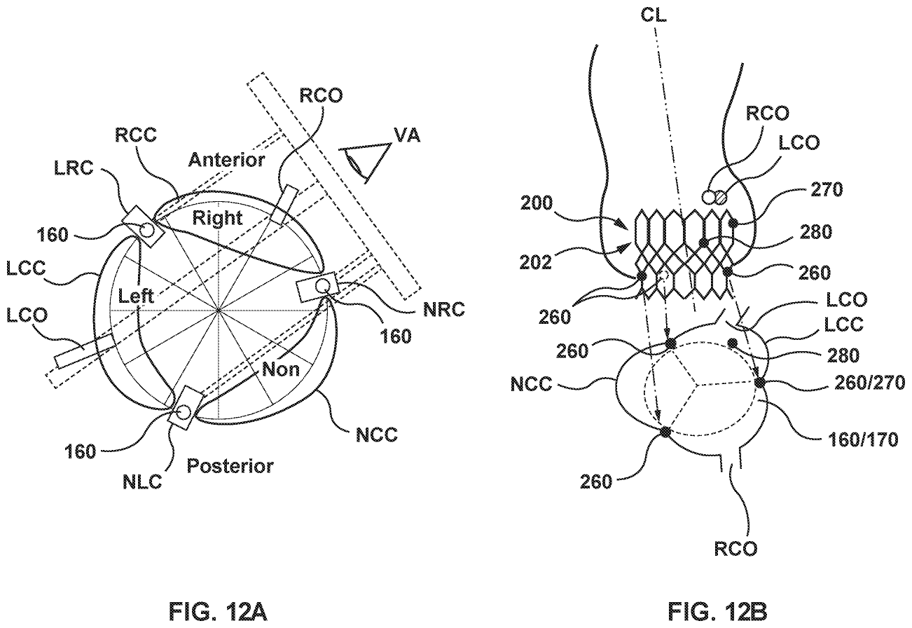

As noted above, imaging systems such as fluoroscopic imaging systems used during transcatheter aortic valve replacement procedures generally include a C-arm gantry that enables different viewing angles of the native aortic valve. One particular viewing angle is a “cusp overlap view” or “cusp overlap viewing angle”. In the cusp overlap view, as shown in FIG. 12A, the viewing angle VA of the imaging system is such that the right coronary cusp RCC and the left coronary cusp RCC overlap each other. FIG. 12B shows a schematic representation of a fluoroscopic image using the cusp overlap view. The image may also be referred to as the “cusp overlap viewing angle image” or the “cusp overlap view image”. In FIG. 12B, the right side shows the right coronary cusp RCC and the left coronary cusp LCC aligned with each other, i.e., they overlap. In the cusp overlap view, the non-coronary cusp NCC is to the left of the right coronary cusp RCC and the left coronary cusp LCC. Thus, the left side of FIG. 12B represents the non-coronary cusp NCC. FIG. 12B also shows example locations of the left main coronary ostium LCO and the right coronary ostium RCO, although these would not generally be visible in a fluoroscopic image.

With the above understanding of the cusp overlap view and the inflow markers 260 of the transcatheter heart valve prosthesis 200 as described above, a system and method for rotationally aligning the transcatheter heart valve prosthesis 200 will now be described. As known to those skilled in the art, the transcatheter heart valve prosthesis 200 may be delivered percutaneously via femoral access. In particular, in the example of a balloon-expandable transcatheter heart valve prosthesis, e.g. the transcatheter heart valve prosthesis 200, the prosthesis is radially crimped to a radially compressed configuration and disposed on a balloon of a balloon catheter, for example. Characteristics of a patient's native anatomy may be determined prior to starting the procedure, such as by a CT scan. Using this planning CT, a determination may be made prior to the procedure regarding orientation of the delivery system, and hence the transcatheter heart valve prosthesis, when delivering the transcatheter heart valve prosthesis. For example, and not by way of limitation, a feature of the delivery system may be aligned with a feature of the transcatheter heart valve prosthesis 200 such that there is confidence that the transcatheter heart valve prosthesis 200 will be properly rotationally oriented when delivered to the native aortic valve, i.e., the transcatheter heart valve prosthesis 200 is oriented such as not to block access to the left main coronary artery ostium LCO and the right coronary ostium RCO. Using pre-procedure CT, the orientation of a feature of the delivery system that has a known relationship to a feature of the transcatheter heart valve prosthesis, such as one of the commissures of the transcatheter heart valve prosthesis 100, may be further defined by the specific patient anatomy. Thus, using pre-procedure planning, a prediction can be made regarding a preferred orientation of the delivery system to reduce the risk of coronary artery obstruction.

Further, during the procedure, the cusp overlap view and inflow markers 260 may be used to confirm that the transcatheter heart valve prosthesis 200 is rotationally aligned such as to not cause coronary obstruction. The transcatheter heart valve prosthesis 200 may be delivered via a balloon catheter past the native valve leaflets/cusps until the outflow markers 260 are aligned with the annulus of the native heart valve, as explained above. To confirm rotational orientation, the C-arm gantry of the imaging system is located in the cusp overlap view. As shown in FIG. 12B, if two of the inflow markers 260 of the transcatheter heart valve prosthesis 100 can be seen left of the centerline CL and one of the inflow markers 260 can be seen right of the centerline CL, the transcatheter heart valve prosthesis 200 is properly rotationally oriented such as to avoid coronary artery obstruction and achieve commissural alignment. In particular, it is desirable for the two inflow markers 260 left of the centerline CL to be substantially aligned. As used herein, “substantially aligned” means within circumferentially within one cell or 30 degrees of each other. It is noted that left and right as used regarding FIG. 12B and other fluoroscopy illustrations is with respect to the fluoroscopy image/illustration, not anatomical left and right. FIG. 12B also shows the projection of the inflow markers 260 onto an idealized normal view of the native aortic valve, which would not be part of the fluoroscopic image.

If two of the inflow markers 260 are not seen left of the centerline CL of the fluoroscopy image, and/or two of the inflow markers 260 are seen to the right of the centerline CL of the fluoroscopy image, then the transcatheter heart valve prosthesis 200 may be rotated until there are two inflow markers 260 to the left side of the image in in the cusp overlap view. If the transcatheter heart valve prosthesis 200 needs to be rotated, it may be rotated by rotating a handle of the delivery system. Non-limiting examples of delivery systems that can be rotated at the handle to rotate a distal end of the delivery system are shown and described in U.S. provisional patent application No. 63/129,194, filed Dec. 22, 2020, the contents of which are incorporated by reference herein in their entirety, or any other suitable delivery system.

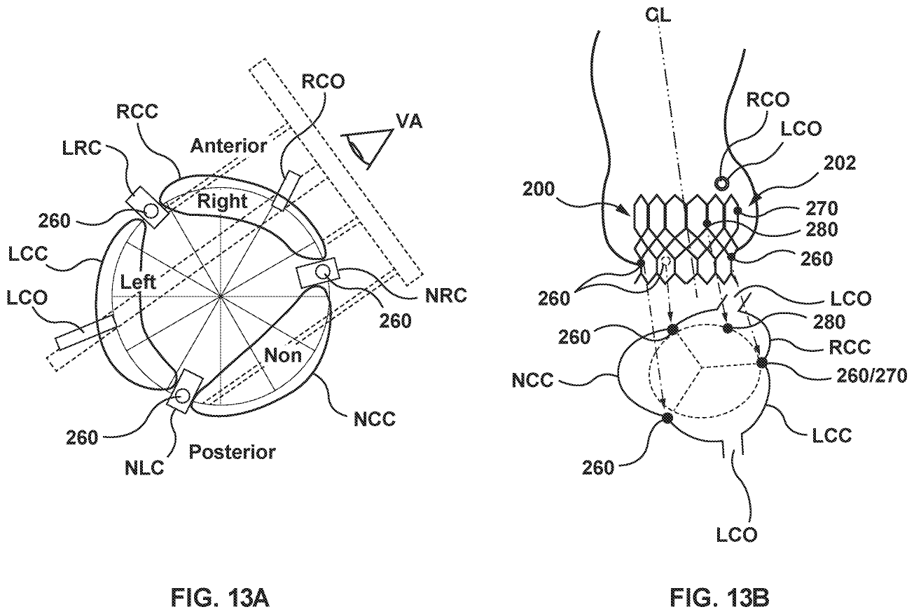

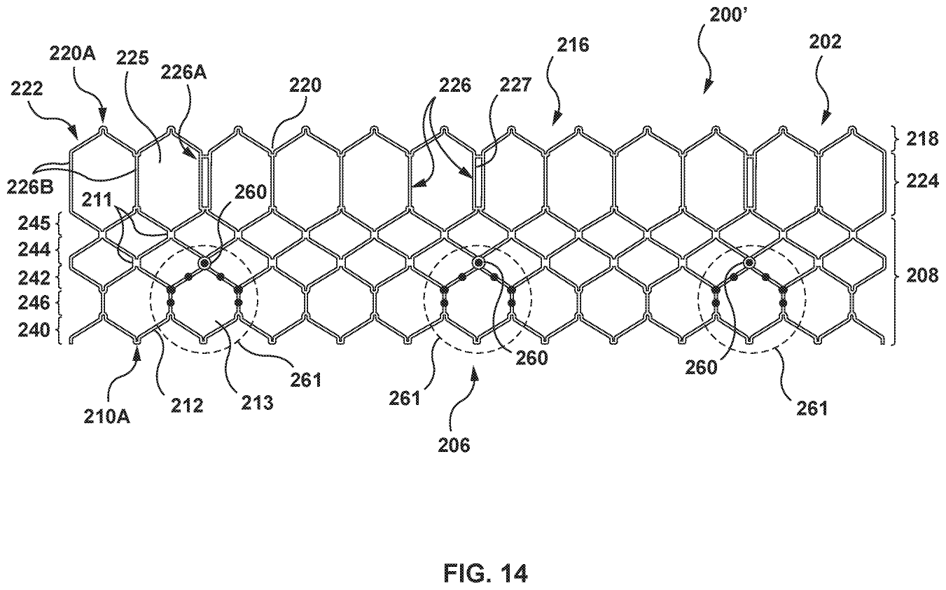

Another viewing angle is referred to herein as the “coronary overlap view” or the “coronary overlap viewing angle”. In the coronary overlap view, during the pre-procedure CT work-up, the location of the coronary ostia (i.e., the openings of the coronary arteries into the sinus of the native aortic valve) are located. Using these locations, it can be determined the proper angle of the C-arm of the imaging system such that the coronary ostia overlap. For example, FIG. 13A shows an example idealized native aortic valve (e.g. the native commissures are 120 degrees apart), with the location of the left coronary ostium LCO and right coronary ostium RCO marked. As shown in FIG. 13A, the viewing angle VA of the imaging system is selected such that the right coronary artery ostium RCO and the left main coronary artery ostium LCO overlap each other. As noted above, patient anatomies vary from this idealized representation. FIG. 13B shows a schematic representation of a fluoroscopic image with the viewing angle set for the coronary overlap view. This image is also referred to as the “coronary overlap viewing angle image” or the “coronary overlap view image”. It is noted that although shown in FIG. 13B for reference, the coronary arteries are not generally visible in the fluoroscopic image, but by using the coronary overlap view, it is known where the coronary ostia are located. Further, because the coronary ostia are aligned in the coronary overlap view, they are commonly located in the 2-Dimensional view of the fluoroscopic image (i.e., one is behind the other). Therefore, knowing the location of the coronary ostia, a clinician can check the location of the commissures of the valve structure of the transcatheter heart valve prosthesis 200 to ensure that none of the prosthetic commissures are aligned or in near alignment with the coronary ostia. In particular, with the transcatheter heart valve prosthesis 200, the commissure posts 226A are aligned with the prosthetic commissures, and the inflow markers 260 are aligned with the commissure posts 226A. Therefore, the coronary overlap view and the inflow markers 260 can be used to avoid coronary obstruction.

As explained above, the planning CT can be used to make a determination prior to the procedure to best orient the delivery system such that rotation of the delivery system at the target site may not be necessary. Further, during the procedure, the coronary overlap view and the inflow markers 260 may be used to confirm that the transcatheter heart valve prosthesis 200 is rotationally aligned such as to not cause coronary obstruction. As explained above, the delivery system is advanced past the native valve leaflets/cusps until the inflow markers 260 are aligned with the annulus of the native heart valve for depth alignment.

For rotational alignment, with the imaging system in the coronary overlap view, if two inflow markers 260 are shown left of the center-line CL of the image and one of the inflow markers 260 is shown to the right of the center-line CL of the image, as shown in FIG. 13B, then the transcatheter heart valve prosthesis 200 is properly rotationally aligned such as to avoid coronary blockage and improve commissural alignment. In particular, it is desirable for the two inflow markers 260 left of the centerline CL to be substantially aligned. As used herein, “substantially aligned” means within circumferentially within one cell or 30 degrees of each other.

It is also noted that using the planning CT and the coronary overlap view, the location of the coronary artery ostia can be marked on the imaging screen. In such an example, viewing the inflow markers 260 in the coronary overlap view as compared to the marked location of coronary artery ostia can confirm proper rotational orientation to avoid coronary artery obstruction.

If two of the inflow markers 260 are not seen left of the centerline CL of the fluoroscopy image, and/or two of the inflow markers 260 are seen to the right of the centerline CL of the fluoroscopy image, and/or an inflow marker 260 is seen aligned with the marked location of the coronary artery ostia, then the transcatheter heart valve prosthesis 200 may be rotated until there are two inflow markers 260 to the left side of the image in in the coronary overlap view. If the transcatheter heart valve prosthesis 200 needs to be rotated, it may be rotated by rotating a handle of the delivery system. Non-limiting examples of delivery systems that can be rotated at the handle to rotate a distal end of the delivery system are shown and described in U.S. provisional patent application No. 63/129,194, filed Dec. 22, 2020, the contents of which are incorporated by reference herein in their entirety, or any other suitable delivery system.

The outflow markers 270, 280 can also be used in the coronary overlap view and/or the cusp overlap view to mark the location of the left main coronary artery ostium LCO. As described above, FIG. 13B shows a schematic representation of a fluoroscopic image with the viewing angle set for the coronary overlap view. In addition to using the inflow markers 260 for rotational alignment, the outflow markers 270, 280 may also be used to assist in rotational alignment and to mark the location of the left main coronary artery ostium LCO. In particular, as shown in FIG. 13B, the first outflow marker 270 is axially aligned with the inflow marker 260 to the extreme right in the coronary overlap view image. The second outflow marker 280 is shown in FIG. 13B to the left of the first outflow marker 270. Due to the location of the second outflow marker 280 relative to the first outflow marker 270, it is known that in the image of FIG. 13B the second outflow marker 280 is located in the rear portion of the native heart valve relative to the viewing angle. In other words, although the image in FIG. 13B is two-dimensional, it is known that portions of the tubular transcatheter heart valve prosthesis 200 are towards the front of the native heart valve in the image and portions of the tubular transcatheter heart valve prosthesis 200 are towards the rear of the native heart valve in the image. The location of the first outflow marker 270 and the location of the second outflow marker 280 relative to the first outflow marker 270 indicate that in the coronary overlap view image, the second outflow marker 280 is towards the rear of the native heart valve. As projected onto the normal view also shown in FIG. 13B, this rotational orientation results in the second outflow marker 280 being located at or near the left main coronary artery ostium LCO, as shown in FIG. 13B. Thus, the second outflow marker 280 can serve as a guide for future procedures that require access to the left main coronary artery. In other words, if after implantation of the transcatheter heart valve prosthesis 200, another procedure is required, such as, but not limited to balloon angioplasty or stenting of a coronary artery, the location of the left main coronary artery ostium LCO will be marked by the second outflow marker 280 to assist in guiding a delivery device to the left main coronary artery ostium LCO. Those skilled in the art will recognize that the marking of the left main coronary artery ostium LCO in the cusp overlap view will work the same as described above with respect to the coronary overlap view. Thus, the description of using the second outflow marker 280 to mark the left coronary main coronary artery ostium is incorporated into the description of the cusp overlap view and the outflow markers 270 and 280 are marked in FIG. 12B in the same manner as FIG. 13B.

The transcatheter heart valve prosthesis 200 described above includes three inflow markers 260 and two outflow markers 270, 280. In an alternative embodiment, a transcatheter heart valve prosthesis 200′ may include only the three inflow markers 160, and no outflow markers. FIG. 14 is a laid open, flat illustration of such the transcatheter heart valve prosthesis 200′. All features of the transcatheter heart valve prosthesis 200 are incorporated into the description of the transcatheter heart valve prosthesis 200′. Further, all methods described above with respect to the transcatheter heart valve prosthesis 200 are incorporated into the description of the transcatheter heart valve prosthesis 200′, except for use of the outflow markers 270, 280 of the transcatheter heart valve prosthesis 200 for rotational alignment in the three-cusp view, as the transcatheter heart valve prosthesis 200 does not include such outflow markers.

FIG. 14 further includes location zones 261 for the inflow markers 260. The location zones 261 show alternative locations for the inflow markers 260 of the transcatheter heart valve prosthesis 200′. In this embodiment, there still will be only three inflow markers 260. FIG. 14 merely shows alternative locations for each of the inflow markers 260. Further, it is preferable, although not required, that the three locations are consistent. In other words, if one of the inflow markers is at the junction between the first row 240 and the second row and to the left of the commissure post 226A, then the other inflow markers 260 are preferably located at the junction between the first row 240 and the second row 242 and to the left of its respective commissure post 226A. The alternative locations for the inflow markers 260 shown in FIG. 14 can also be used in the embodiment of FIGS. 9A-9B which includes the outflow markers 270, 280.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.

1. A transcatheter heart valve prosthesis comprising:
a stent having a radially compressed configuration for delivery within a vasculature and an expanded configuration for deployment within a native heart valve, wherein the stent comprises an inflow portion comprising a plurality of rows of angled struts, an outflow portion including a single row of angled struts, exactly three commissure posts extending between the inflow portion and the outflow portion, and exactly three axial struts extending between the inflow portion and the outflow portion, wherein a single axial strut of the exactly three axial strut is located circumferentially between a corresponding pair of the commissure posts such that there is a single axial strut of the exactly three axial struts between each pair of commissure posts around a circumference of the stent; and
exactly three inflow markers positioned within the inflow portion of the stent, wherein each of the three inflow markers is axially aligned with one of the three commissure posts,
wherein the inflow markers are configured to be visible relative to the stent in one or more images captured during installation at the native heart valve.

2. The transcatheter heart valve prosthesis of claim 1, wherein the inflow markers are located at a junction between a first row and a second row of the plurality of rows of angled struts of the inflow portion.

3. The transcatheter heart valve prosthesis of claim 1, wherein each of the three inflow markers is spaced a first distance from an inflow end of the stent, the first distance being such that the three inflow markers are configured to be disposed at the annulus of the native heart valve.

4. (canceled)

5. The transcatheter heart valve prosthesis of claim 1, further comprising:
a first outflow marker disposed on a first commissure post of the exactly three commissure posts; and
a second outflow marker disposed on a first axial strut of the exactly three axial struts, wherein the first axial strut is disposed between the first commissure post and a second commissure post of the exactly three commissure posts adjacent to the first commissure post.

6. The transcatheter heart valve prosthesis of claim 5, wherein the second outflow marker is disposed counter-clockwise from the first outflow marker when viewed from an outflow end of the stent.

7. The transcatheter heart valve prosthesis of claim 5, wherein the second outflow marker is axially offset from the first outflow marker.

8. The transcatheter heart prosthesis of claim 7, wherein the second outflow marker is disposed upstream of the first outflow marker.

9. The transcatheter heart valve prosthesis of claim 7, wherein the second outflow marker is disposed downstream of the first outflow marker.

10. The transcatheter heart valve prosthesis of claim 5, wherein the second outflow marker is circumferentially offset from the first outflow marker by approximately 60 degrees.

11. A transcatheter heart valve prosthesis of comprising:
a stent having a radially compressed configuration for delivery within a vasculature and an expanded configuration for deployment within a native heart valve, wherein the stent comprises an inflow portion comprising a plurality of rows of angled struts, an outflow portion including at least one row of angled struts, three commissure posts extending between the inflow portion and the outflow portion, at least nine axial struts extending between the inflow portion and the outflow portion, wherein three axial struts are located circumferentially between a corresponding pair of the commissure posts such that there are three axial struts between each pair of commissure posts around a circumference of the stent;
three inflow markers positioned within the inflow portion of the stent, wherein each of the three inflow markers is axially aligned with one of the three commissure posts;
a first outflow marker disposed on a first commissure post of the three commissure posts; and
a second outflow marker disposed on a first axial strut of the at least nine axial struts, wherein the first axial strut is disposed between the first commissure post and a second commissure post of the three commissure posts adjacent to the first commissure post,
wherein the inflow markers are configured to be visible relative to the stent in one or more images captured during installation at the native heart valve.

12. (canceled)

13. The transcatheter heart valve prosthesis of claim 11, wherein the second outflow marker is disposed counter-clockwise from the first outflow marker when viewed from an outflow end of the stent.

14. The transcatheter heart valve prosthesis of claim 11, wherein the second outflow marker is axially offset from the first outflow marker.

15. The transcatheter heart prosthesis of claim 14, wherein the second outflow marker is disposed upstream of the first outflow marker.

16. The transcatheter heart valve prosthesis of claim 14, wherein the second outflow marker is disposed downstream of the first outflow marker.

17. The transcatheter valve prosthesis of claim 12, wherein the second outflow marker is circumferentially offset from the first outflow marker by approximately 60 degrees.

18. A method for rotationally aligning a transcatheter heart valve prosthesis within a native heart valve, the method comprising:
percutaneously delivering the transcatheter heart valve prosthesis to the native heart valve, wherein the transcatheter heart valve prosthesis includes at least one marker substantially aligned with a commissure of the transcatheter heart valve prosthesis;
receiving an image of the transcatheter heart valve prosthesis within the native heart valve;
determining, based on the image and the at least one marker, whether the transcatheter heart valve prosthesis is in a desired rotational orientation; and
if the at least one marker in the viewing angle image indicates that the transcatheter heart valve prosthesis is not in the desired rotational orientation, rotating the transcatheter heart valve prosthesis until the transcatheter heart valve prosthesis is in the desired rotational orientation.

19. The method of claim 18, wherein the at least one marker is disposed adjacent an inflow end of the transcatheter heart valve prosthesis.

20. The method of claim 18, wherein the viewing angle is a cusp overlap viewing angle and the image is a cusp overlap viewing angle image.

21. The transcatheter heart valve prosthesis of claim 1, further comprising:
exactly three disks positioned in the inflow portion of the stent, each of the exactly three disks axially aligned with a corresponding one of the exactly three axial posts, wherein the exactly three inflow markers and the exactly three disks are disposed a first distance from an inflow end of the stent.
